(19) United States
(12) Patent Application Publication
(10) Pub. No.: US 2023/0414754 A1
(43) Pub. Date: Dec. 28, 2023

(54) CONDITIONAL CONTROL OF UNIVERSAL CAR T CELLS THROUGH STIMULUS-REACTIVE ADAPTORS

(71) Applicant: UNIVERSITY OF PITTSBURGH-OF THE COMMONWEALTH SYSTEM OF HIGHER EDUCATION, Pittsburgh, PA (US)

(72) Inventors: Jason Jakob LOHMUELLER, Pittsburgh, PA (US); Alexander DEITERS, Pittsburgh, PA (US)

(21) Appl. No.: 18/035,590

(22) PCT Filed: Nov. 5, 2021
(86) PCT No.: PCT/US2021/058325

§ 371 Date: May 5, 2023

Related U.S. Application Data
(60) Provisional application No. 63/110,194, filed on Nov. 5, 2020.

(51) Int. Cl. A61K 39/00

(52) U.S. Cl. CPC A61K 39/4633; A61K 39/4631; A61K 39/4611; A61K 2239/26; A61K 2239/24

(57) ABSTRACT

Disclosed are compositions and methods related to the construction and use of conditional universal synthetic notch (synNotch) receptors and conditional chimeric antigen receptor (CAR) T cells. Also disclosed herein are methods of using said synNotch receptors in the construction of engineered cells. It is contemplated herein that the disclosed synNotch receptors and engineered cells can be used in regenerative medicine. Additionally disclosed herein are methods of using the disclosed synNotch, CAR T cells, and engineered cells in the treatment of cancer, autoimmune disease, autoinflammatory disease, and infectious disease.

This application claims the benefit of U.S. Provisional Application No. 63/110,194, filed on Nov. 5, 2020, which is incorporated herein by reference in its entirety.

This invention was made with government support under RO1 GM142007, R35 CA210039 and R21 AI130815 awarded by the National Institutes of Health. The government has certain rights in the invention.

### I. BACKGROUND

1. Despite of the promise that CAR T cells might have in treating cancer patients there are several limitations to the generalized clinical application of CART cells, First, since no single tumor antigen is universally expressed by all cancer types, say in CAR needs to be constructed for each tumor antigen to be targeted. Second, the financial cost and labor-intensive tasks associated with identifying and engineering says against a variety of tumor antigens poses a major challenge. Third, tumor antigens targeted by CAR could be down-regulated or mutated in response to treatment resulting in tumor evasion. Since current CAR T cells recognize only one target antigen, such changes in tumors negate the therapeutic effects. Therefore, the generation of CAR T cells capable of recognizing multiple tumor antigens is highly desired, Finally, their implementation has been limited by difficulties in attaining cancer cell-specificity through single antigen targeting leading to unwanted ON-target/OFF-disease toxicities and toxicities from overactive cells. Modifications to existing CAR T cell systems that address and overcome the hurdles currently preventing development of the systems into effective means of in vivo treatment are therefore needed.

### II. SUMMARY

2. Disclosed are methods and compositions related to conditional universal chimeric antigen receptor (CAR) systems, conditional universal synthetic Notch (synNotch) receptors, and methods of their use.

3. In one aspect, disclosed herein are conditional universal chimeric antigen receptor (CAR) system, comprising i) a CAR, comprising a receptor that targets a tag ligand (such as, for example, benzylguanine (BG), Benzylcytosine (BC), chloroalkane, fluoresceine (FITC), SpyTag, leucine-zipper, La-SS-B, CD19, anti-folate receptor antibody, Fc domain, peptide neoepitope (PNE), and biotin) on a conditional adaptor molecule, a hinge domain (such as, for example, a CD8α domain, CD28 hinge, or modified IgG hinges with a deletion or modification of the CH2 and/or CH3 domain), and a signaling domain (such, as for example a DAP10, NKG2D, NKG2C, NKp44, CD28, CD27, Meg10, CD32, CD16, 2B4, or CD3ζ signaling domain); and ii) a conditional adaptor molecule comprising an antigen recognition element (such as, for example an antibody, antigen recognizing fragment thereof, a protein binding domain. lectin, DNA aptamer, RNA aptamer, a small molecule ligand for cell surface receptor, or a peptide/protein ligand for natural protein receptor), a stimulus reactive group, and a tag ligand (such as, for example, benzylguanine (BG), benzylcytosine (BC), chloroalkane, fluorescein (isothiocyanate), SpyTag, leucine-zipper, La-SS-B, CD19, anti-folate receptor antibody, Fc domain, peptide neoepitope (PNE), or biotin).

4. Also disclosed herein are conditional universal CAR systems of any preceding aspect, wherein the conditional adaptor molecule comprises NHS-ester conjugation, disulfide re-stapling approaches (including, but not limited to bis-sulfone conjugation and other disulfide re-stapling approaches), glycan conjugating chemistry, a recombinant antibody with tag ligand incorporation (such as, for example, BG or BC incorporation) through one or more short peptide tags, sortase mediated ligation, chemical ligation, split inteins, THIOMABs, and/or unnatural amino acids. In some aspect, the antigen recognition element is covalently linked to the universal adaptor molecule.

5. In one aspect, disclosed herein are conditional universal CAR systems of any preceding aspect, wherein the antigen recognition element comprises rituximab, FMC63, herceptin, cetuximab, nimatuzumab, panitumumab, omalizumab, tositumomab, trastuzumab, gemtuzumab, alemtuzumab, bevacuzimab, or an antigen-binding fragment of any one thereof.

6. Also disclosed herein are conditional universal CAR systems of any preceding aspect, wherein the stimulus to which the stimulus reactive group is reactive comprises a light (such as, for example, 365, 405, 544, 780 nm), enzyme (such as, for example, legumain, matrix metalloproteinase, pyridoxal kinase (PDXK), aldehyde dehydrogenase 7 family, member A1, (ALDH7A1), lipase C, hepatic type (LIPC), poly(ADP-ribose) polymerase 1 (PARP1), pyruvate kinase M2 (PKM2), phosphoglycerate kinase 1 (PGK1), ketohexokinase-A (KHK-A), hexokinases (HK), nucleoside diphosphate kinase (NDPK or NDK), and 6-phosphofructo-2-kinase/fructose-2,6-biphosphatase 4 (PFKFB4), mitochondrial α-ketoglutarate dehydrogenase (α-KGDH), lysine acetyltransferase 2A (KAT2A), acetyl-CoA synthetase short-chain family member 2 (ACSS2), ATP-citrate lyase (ACLY), pyruvate dehydrogenase complex (PDC), α-ketoglutarate dehydrogenase (α-KGDH), CD39, CD73, or fumarase), small molecule (such as, for example, phosphine or tetrazine), pH, hypoxia, H₂O₂, and/or ROS.

7. In one aspect, disclosed herein are conditional universal CAR systems of any preceding aspect, wherein the stimulus reactive group comprises a cleavable linker (such as, for example, a photocleavable or phosphine-cleavable linker) or a stimulus reactive caging group (for example, a light reactive caging group comprising nitrobenzyl, coumarin, BODIPY, or cyanin) that blocks the CAR from binding to the conditional adaptor molecule.

8. Also disclosed herein are conditional universal CAR systems of any preceding aspect, further comprising one or more co-stimulation domains (such as, for example, CD27, CD28, ICOS, 4-1BB, or OX40).

9. In one aspect, disclosed herein are conditional universal CAR systems of any preceding aspect, wherein tag ligand targeting CAR is comprised on a CAR T cell, CAR NK cell, CAR NK T cell, CAR B cell, or CAR macrophage.

10. In one aspect, disclosed herein are conditional universal synthetic Notch (synNotch) receptor systems comprising a conditional adaptor molecule comprising an antigen biding domain, a stimulus reactive group and a tag (such as, for example, benzylguanine (BG), benzylcytosine (BC), chloroalkane, FITC, SpyTag, leucine-zipper, La-SS-B, CD19, anti-folate receptor antibody, Fc domain, peptide neoepitope (PNE), and biotin), a synthetic Notch receptor with a tag reactive domain, a notch core comprising one or more cleavage sites, and one or more transcription factors.

11. Also disclosed herein are conditional universal synNotch receptor systems of any preceding aspect, wherein the adaptor molecule comprises NHS-ester conjugation, disulfide re-stapling approaches (including, but not limited to bis-sulfone conjugation and other disulfide re-stapling approaches), glycan conjugating chemistry, a recombinant antibody with tag incorporation (such as, for example, BG or BC incorporation) through one or more short peptide tags, sortase mediated ligation, chemical ligation, split inteins, THIOMABs, and/or unnatural amino acids.

12. In one aspect, disclosed herein are conditional universal synNotch receptors of any preceding aspect, wherein the stimulus to which the stimulus reactive group is reactive comprises a light (such as, for example, 365, 405, 544, 780 nm), enzyme (such as, for example, legumain, matrix metalloproteinase, pyridoxal kinase (PDXK), aldehyde dehydrogenase 7 family, member A1, (ALDH7A1), lipase C, hepatic type (UPC), poly(ADP-ribose) polymerase 1 (PARP1), pyruvate kinase M2 (PKM2), phosphoglycerate kinase 1 (PGK1), ketohexokinase-A (KHK-A), hexokinases (HK), nucleoside diphosphate kinase (NDPK or NDK), and 6-phosphofructo-2-kinase/fructose-2,6-biphosphatase 4 (PFKFB4), mitochondrial α-ketoglutarate dehydrogenase (α-KGDH), lysine acetyltransferase 2A (KAT2A), acetyl-CoA synthetase short-chain family member 2 (ACSS2), ATP-citrate lyase (AUX), pyruvate dehydrogenase complex (PDC), α-ketoglutarate dehydrogenase (α-KGDH), CD39, CD73, or fumarase), small molecule (such as, for example, phosphine or tetrazine), pH, hypoxia, H₂O₂, and/or reactive oxygen species (ROS).

13. Also disclosed herein are conditional universal synNotch receptors of any preceding aspect, wherein the stimulus reactive group comprises a cleavable linker (such as, for example, a photocleavable or phosphine cleavable linker) or a stimulus reactive caging group (for example, a light reactive caging group comprising nitrobenzyl, coumarin, BODIPY, or cyanin) that blocks the CAR from binding to the conditional adaptor molecule.

14. In one aspect, disclosed herein are conditional universal synNotch receptors of any preceding aspect, wherein the transcription factor comprises Gal4-VP64, Gal4-VP16, TetR-VP64, or LacI-VP64.

15. Also disclosed herein are conditional universal synNotch receptors of any preceding aspect, further comprising an antigen recognition element (such as, for example an antibody, antigen recognizing fragment thereof, a protein binding domain, lectin, DNA aptamer, NA aptamer, a small molecule ligand for cell surface receptor, or a peptide/protein ligand for natural protein receptor); wherein the antigen recognition element is or can become covalently linked to the conditional universal adaptor molecule.

16. In one aspect, disclosed herein are conditional universal synNotch receptors of any preceding aspect, wherein the antigen recognition element comprises rituximab, FMC63, herceptin, cetuximab, nimotuzumab, panitumumab, omalizumab, tositumomab, trastuzurnab, gemtuzumab, al emtuzumab, bevacuzimab or an antigen-binding fragment of any one thereof.

17. Also disclosed herein are engineered cells (such as, for example, an immune cell (including, but not limited to T cells, NK cells, NK T cells, B cells, and macrophage), a neuron, an epithelial cell, and endothelial cell, or a stem cell) comprising the conditional universal CAR systems of any preceding aspect and/or the conditional universal synNotch of any preceding aspect.

18. In one aspect, disclosed herein are engineered cells of any preceding aspect, further comprising a vector comprising a transcriptional response element operatively linked to a promoter driving expression of one or more cell response genes (such as, for example IL-4, IL-10, FASL, IFN-γ, TNF-α, granzyme A. granzyme B, granulysin, and/or perforin); wherein the one or more of the transcription factors on the synNotch receptor are specific for the transcriptional response element.

19. Also disclosed herein are engineered cells of any preceding aspect, wherein one or more transcription factors of the conditional universal synNotch receptor activate expression of one or more native cell response genes (such as, for example, IL-4, IL-10, FASL, IFN-γ, TNF-αgranzyme A. granzyme B, granulysin, and/or perforin).

20. In one aspect, disclosed herein are methods of treating, decreasing, reducing, inhibiting, ameliorating, and/or preventing a cancer and/or metastasis in a subject comprising administering to the subject the conditional universal chimeric antigen receptor (CAR) system of any preceding aspect and/or the engineered cell of any preceding aspect to the subject.

21. Also disclosed herein are methods of treating, decreasing, reducing, inhibiting, ameliorating, and/or preventing a cancer and/or metastasis of any preceding aspect comprising administering to the subject a first CAR system of any preceding aspect and a second conditional universal CAR system of any preceding aspect; wherein the first conditional universal chimeric antigen receptor CAR system comprises a stimulus reactive group comprising stimulus cleavable linker and the second CAR system comprises a stimulus reactive caging group that blocks the CAR from binding to the conditional adaptor molecule. In one aspect, the first and second conditional universal CAR systems are reactive to the same stimulus. In another aspect, the first and second conditional universal CAR systems are reactive to different stimuli.

### III. BRIEF DESCRIPTION OF THE DRAWINGS

22. The accompanying drawings, which are incorporated in and constitute a part of this specification, illustrate several embodiments and together with the description illustrate the disclosed compositions and methods.

23. FIGS. 1A, 1B, and IC show a schematic of antigen receptors. FIG. 1A shows a chimeric antigen receptor (CAR) binding to antigen clusters receptors activating the T cell signaling pathway, FIG. 1B shows synNotch receptor binding to antigen leads to receptor cleavage and release of the transcription factor (purple) to the nucleus to turn on a custom gene program. FIG. 1C show universal CARs binding to a tag ligand molecule (red) on an antibody adaptor that allows for the same receptor to target multiple antigens on the target cell.

24. FIG. 2 shows an overview of OFF-switch adaptors that are generated through light- and small molecule-cleavable benzylguanine (BG) groups to inactivate SNAP receptor activity. ON-switch adaptors allow for receptor signaling activation through BG decaging in response to light, small molecules, low proteases, or reactive oxygen species (ROS). Two adaptors bind to different antigens on the target cell and for AND logic, one adaptor enzymatically decages the BG on the other adaptor, while for NOT logic it cleaves off the BG.

25. FIG. 3 shows a schematic of covalent adaptor SNAP-CAR and SNAP-synNotch receptor function assembly. A benzylguanine (BG) was chemically conjugated to an antibody using the BG-NHS ester. The BG-antibody conjugate then fuses to the cell surface SNAPtag through a self-labeling reaction. This allows for universal re-targeting of CAR and synNotch signaling to any antigen of interest.

26. FIGS. 4A, 4B, 4C, and 4D show that the engineered SNAP-CAR is effective on primary human T cells. FIG. 4A shows a schematic of SNAP-CAR and BG-adaptor mediated. activation, FIG. 4B shows flow cytometry analysis of CD62L T cell activation marker on SNAP-CAR T cells co-incubated with target cell lines (x-axis) and the indicated antibody (1.0 μg/mL), reported as mean fluorescence intensity (MFI). FIG. 4C shows ELISA data for IFNγ production from primary human T cells after the incubations in 4B. FIG. 4D shows specific lysis of target cells by primary human SNAP-CAR T cells and BG-conjugated antibodies. For 4B, 4C, and 4D, multiple ANOVA comparisons were performed. Tukey's HSD was used for post-hoc analysis between antibody conditions. “*” denotes p<0.0001, n=3 independent experiments±s.e.m.

27. FIG. 5A shows the structure of a light-cleavable biotin NHS carbonate.

28. FIG. 5B shows light-cleavable OFF-switch adaptors deactivate mSA2 CAR T cell function. Primary human mSA2-CAR T cells were incubated for 24 h with Raji CD20+ target cells and various concentrations of anti-CD20 OFF-switch adaptor antibody exposed to 365 nm light, T cell activation markers CD69 (increases with activation) and CD62L (decreases with activation) measured by flow cytometry. n=3 biol. replicates.

29. FIGS. 6A and 6B show that small molecule-cleavable OFF-switch adaptor deactivated mSA2 CAR cell function. FIG. 6A shows a biotin adaptor containing an aryl azide that triggers cleavage via a 1,6-elimination in response to a phosphine-induced Staudinger reduction, FIG. 6B shows primary human mSA2-CAR T cells that were incubated for 24 h with Raji CD20+ target cells and increasing concentrations of anti-CD20 OFF-switch adaptor exposed to 2DPBM as indicated. T cell activation markers CD69 and CD62L measured by flow cytometry.

30. FIG. 7A shows a light activated biotin.

31. FIG. 7B shows that the light-triggered ON-switch adaptor anti-CD20 antibody displays biotin on target cell surfaces, measured by streptavidin-APC cell staining, in a light-dependent manner (365 nm).

32. FIG. 7C shows that the light-triggered OFF-switch adaptor anti-CD20 antibody displays light-triggered biotin cleavage on CD20+ Raji target cell surfaces, in a light exposure time-dependent manner (365 nm) measured by streptavidin-APC cell staining by flow cytometry.

33. FIG. 8A shows a phosphine activated biotin.

34. FIG. 8B shows small molecule-triggered ON-switch adaptor anti-CD20 antibody in a 2DPBM-dependent manner (same staining protocol as in FIG. 7).

35. FIG. 8C shows small molecule-triggered OFF-switch adaptor anti-CD20 antibody displays 2DPBM-triggered biotin cleavage on CD20+ Raji target cell surfaces, in a 2DBPM concentration-dependent manner measured by streptavidin-APC cell staining by flow cytometry.

36. FIG. 9 shows the clinical utility of OFF-switch adaptor control. A light-triggered OFF-switch allows for spatiotemporal protection of an anatomical site from CAR T cell toxicity treating a primary tumor and distal metastatic sites, while, small molecule drug-triggered OFF-switch offers rapid, systemic cessation of therapy.

37. FIGS. 10A and 10B show proposed syntheses of BG adaptors capable of site-specific antibody conjugation through (10A) a bis-sulfone or (10B) a dibromopyradizinedione.

38. FIG. 11 shows site-specific labeling of antibodies via re-bridging of disulfides. Disulfides can be reduced using tris-(2-carboxyethyl)phosphine (TCEP) and then re-bridged using established dibromopyridazinediones or bis-sulfone reagents. Antibodies contain four disulfides; thus, four conjugates can be site-specifically added (Only one shown here for clarity).

39. FIGS. 12A and 12B show MTGase-mediated, enzymatic antibody conjugation. FIG. 12A shows the synthesis of substrate for mTG-mediated conjugation. The PEG spacers 13 are commercially available (n=0-6). FIG. 12B shows two conjugates are site-specifically installed (one shown for clarity).

40.

FIGS. 13A and 13B show syntheses of (13A) BG with a phosphine cleavable linker (green) and (13B) a light-cleavable linker (blue).

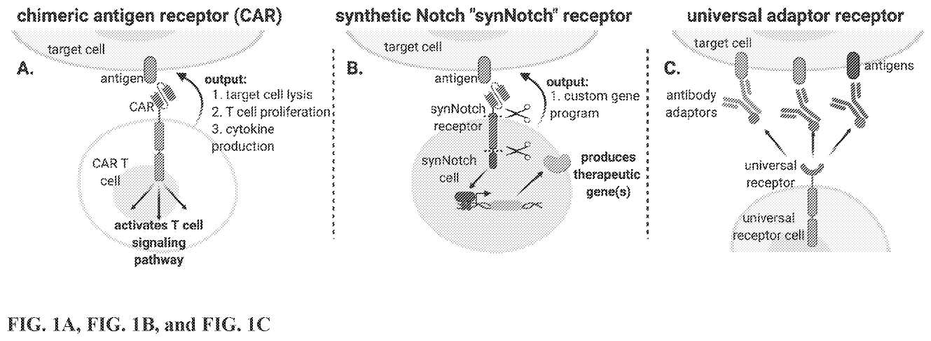

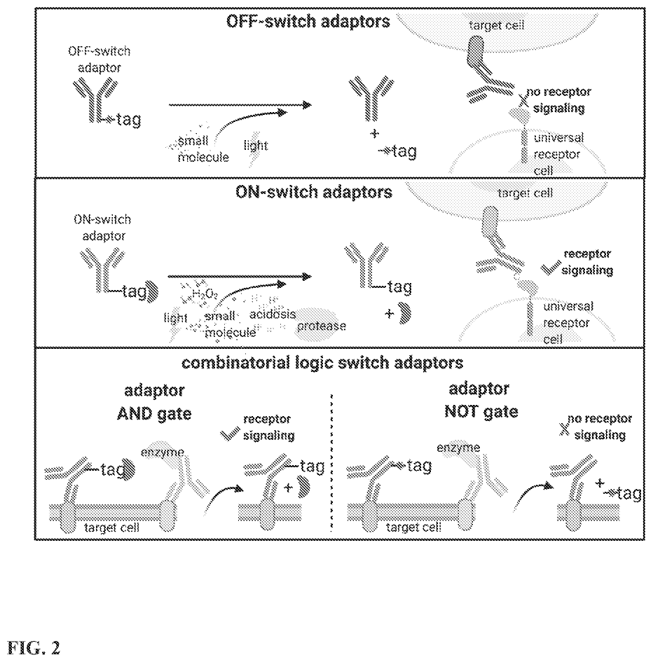

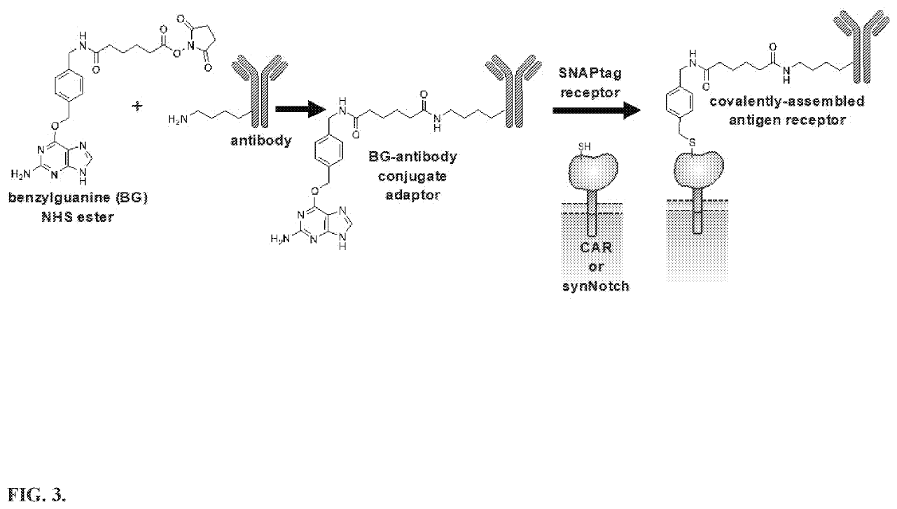

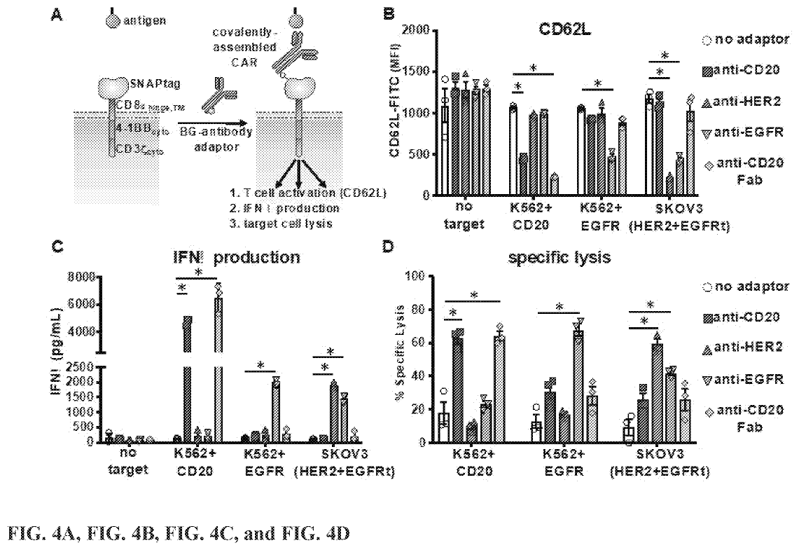

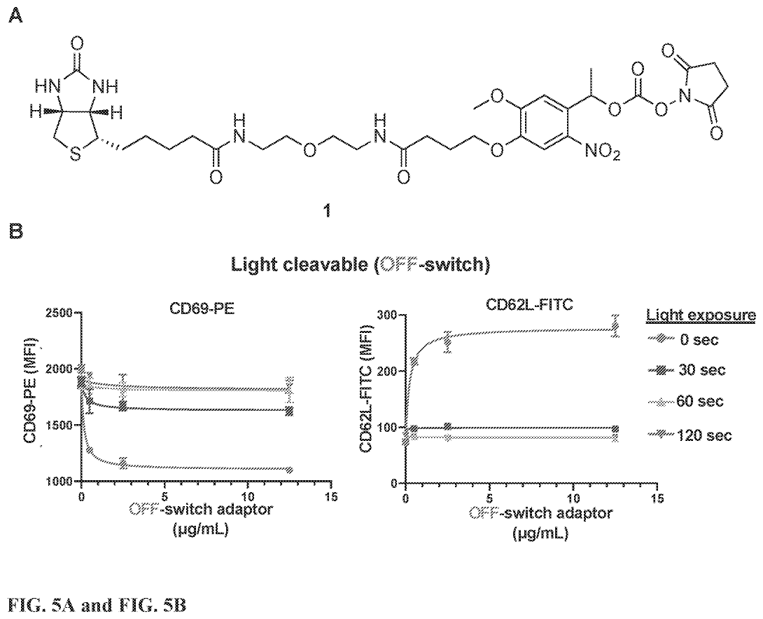

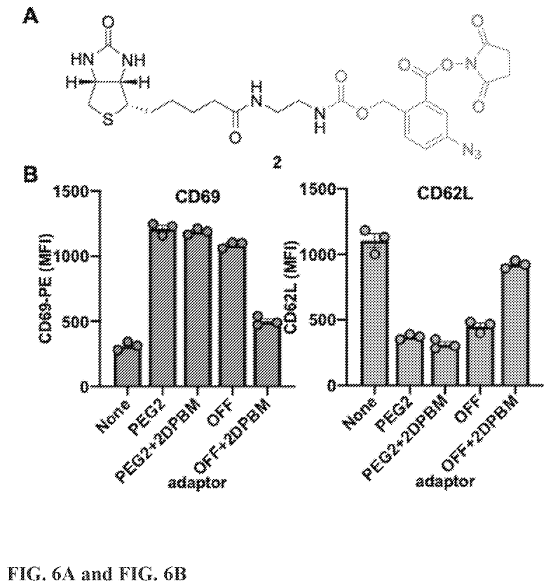

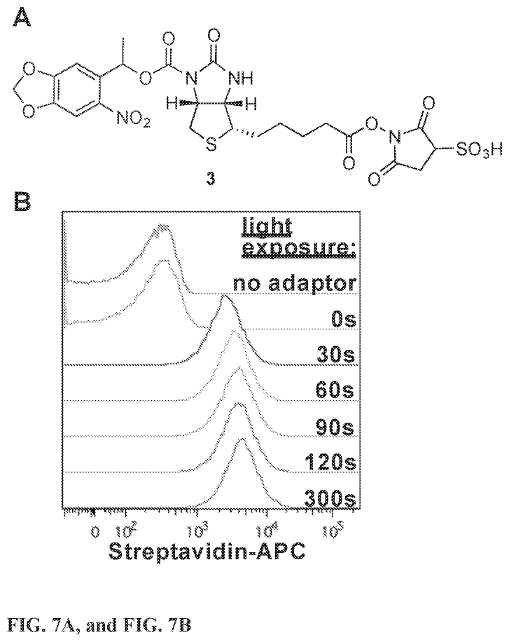

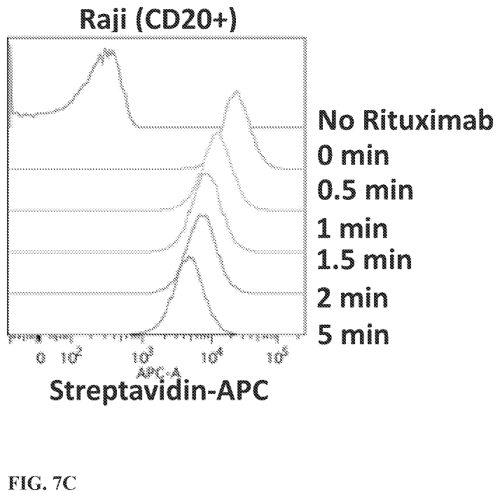

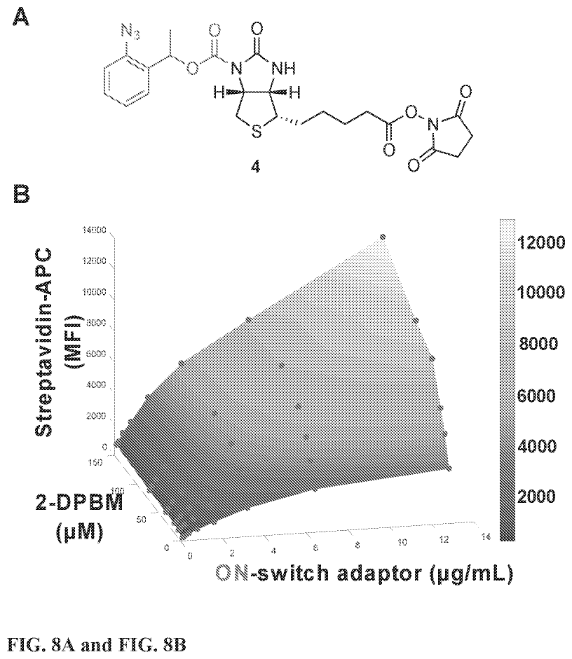

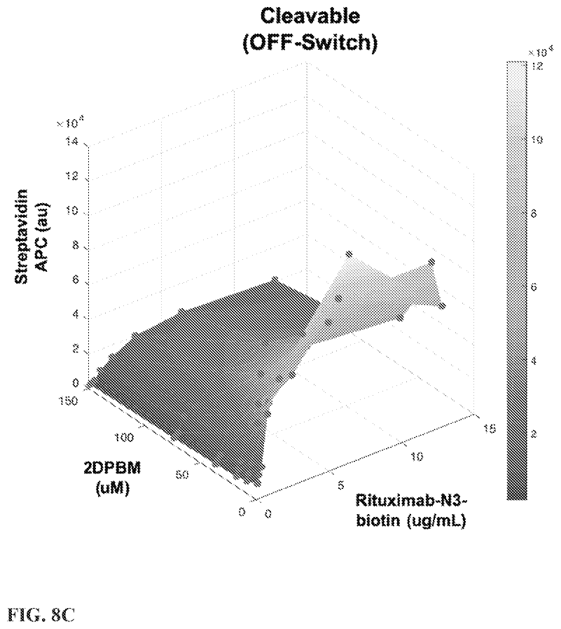

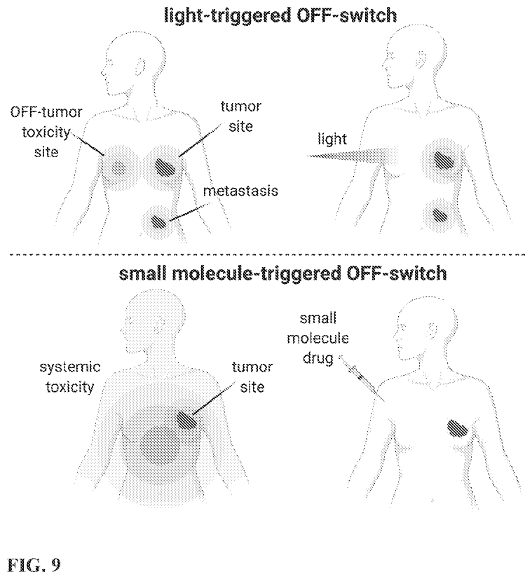

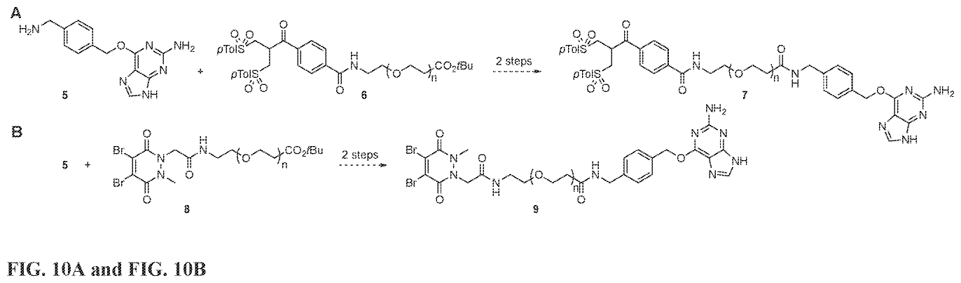

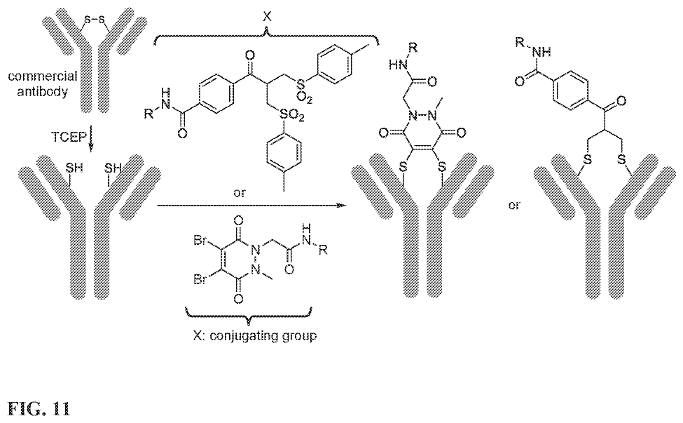

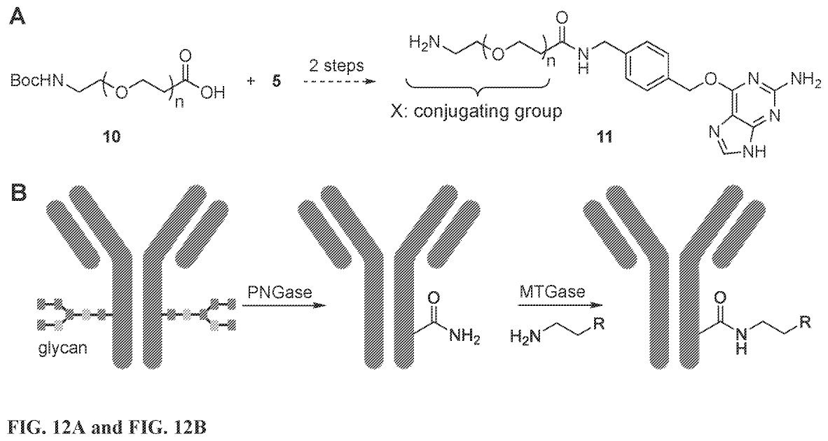

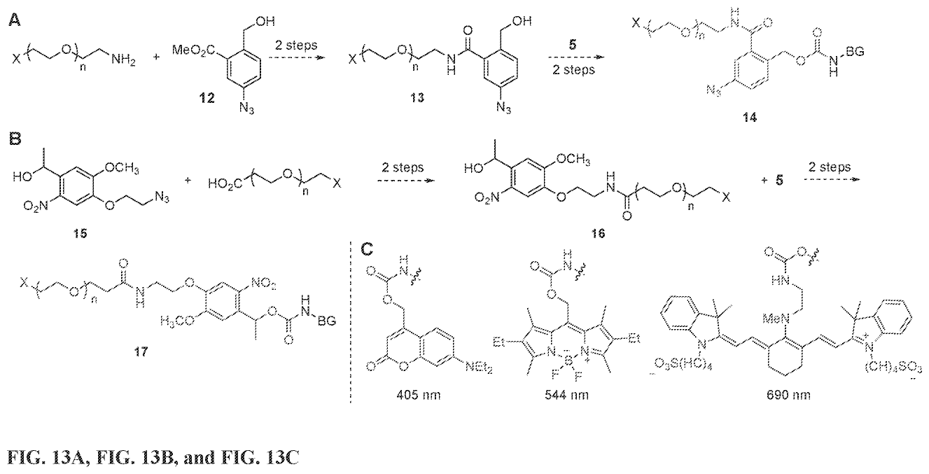

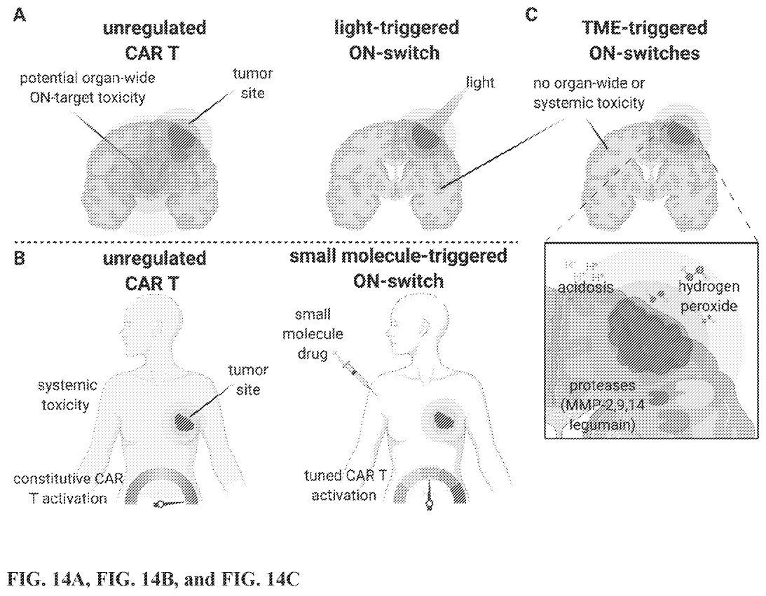

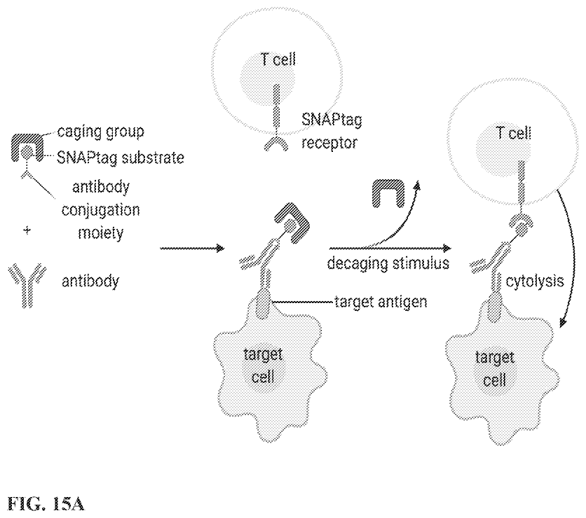

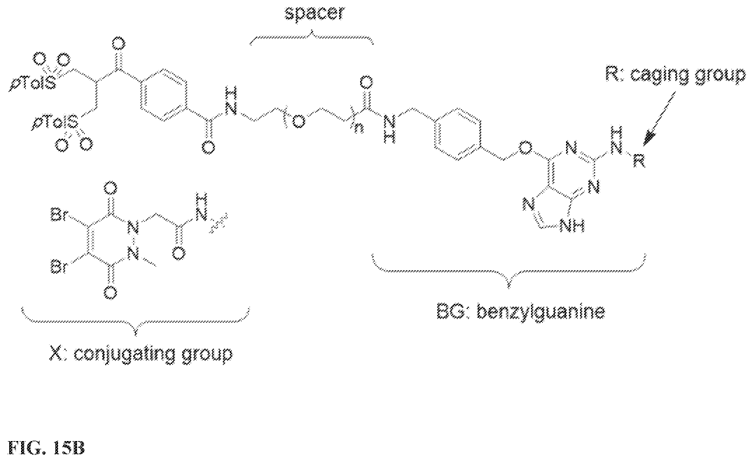

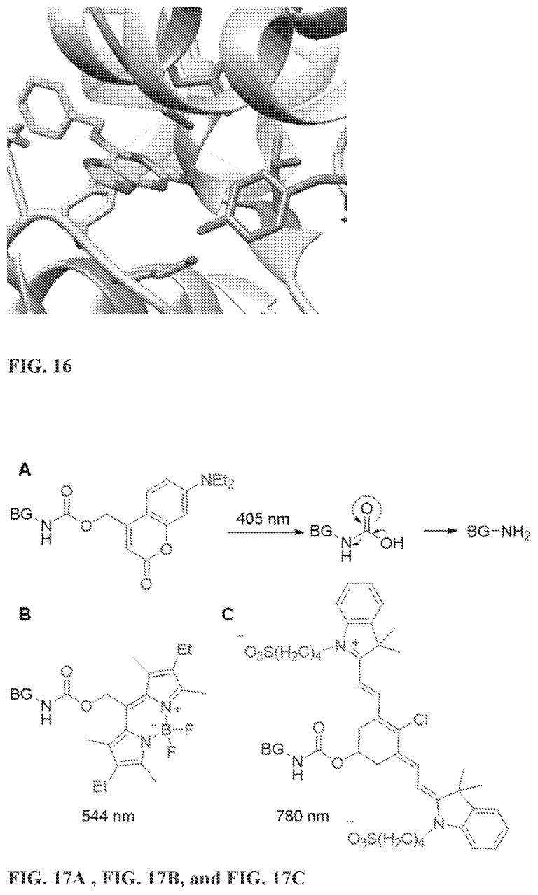

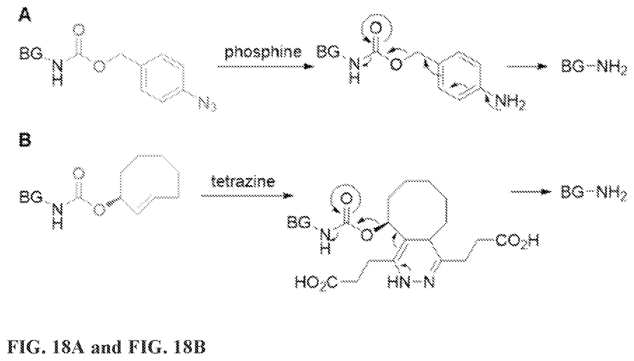

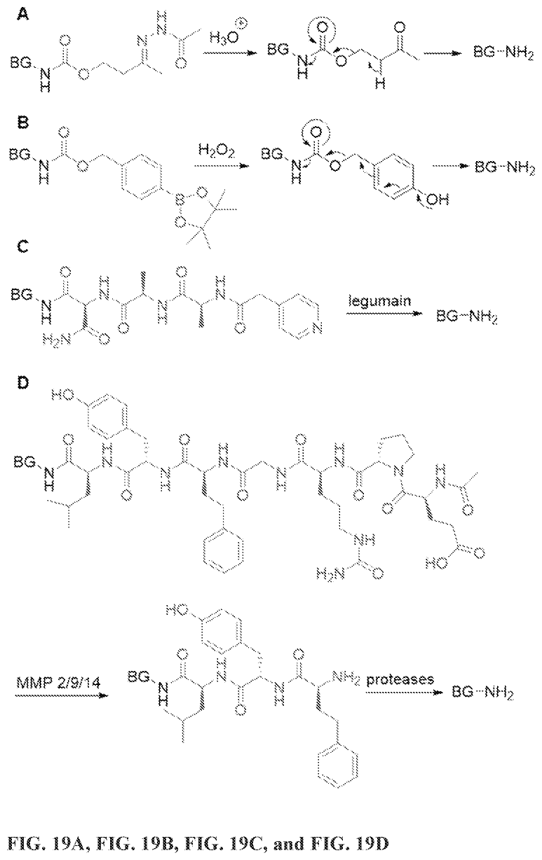

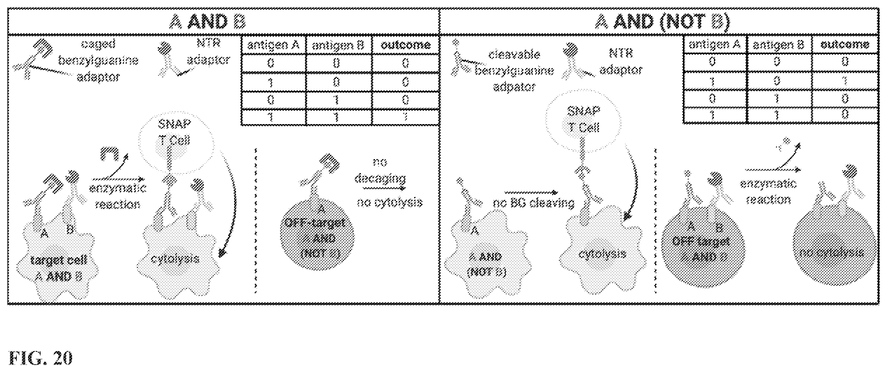

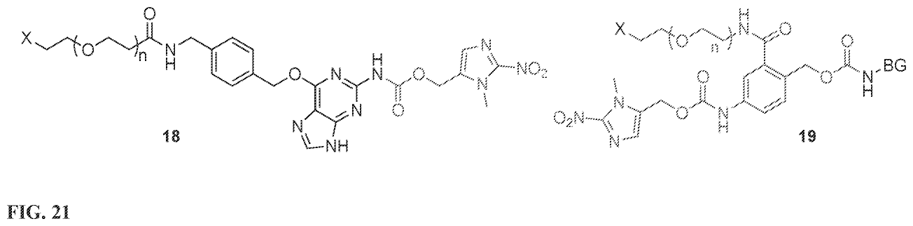

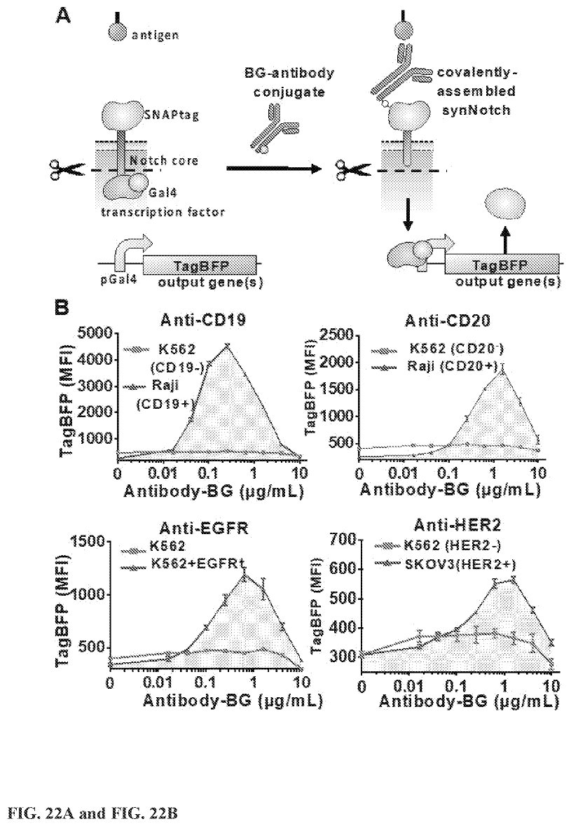

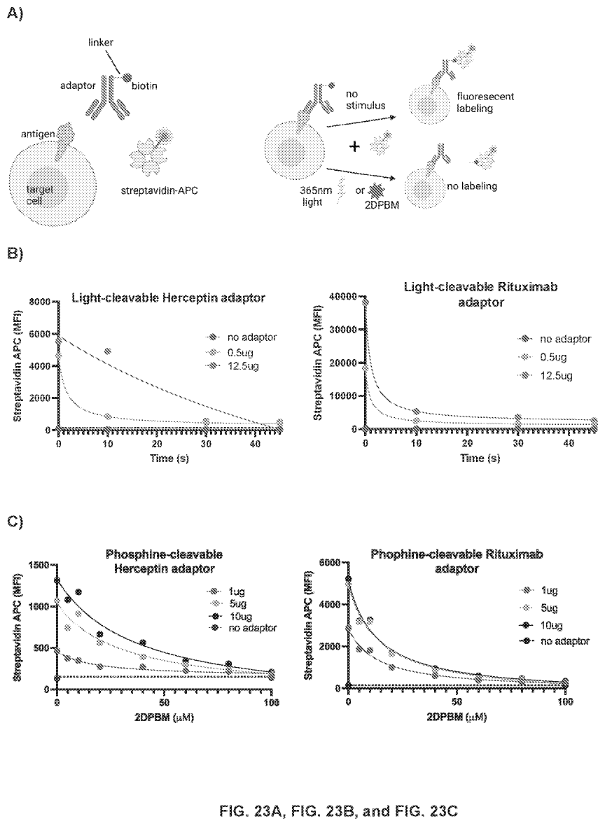

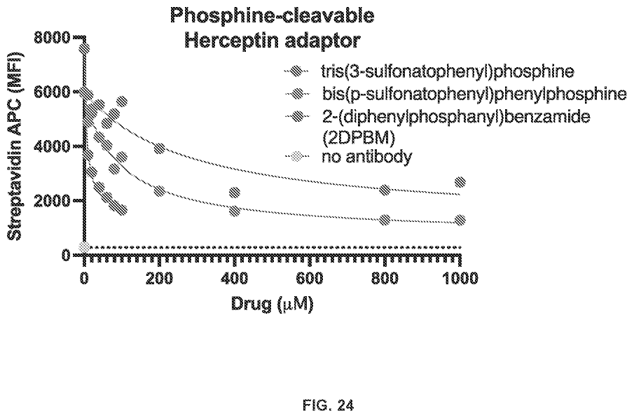

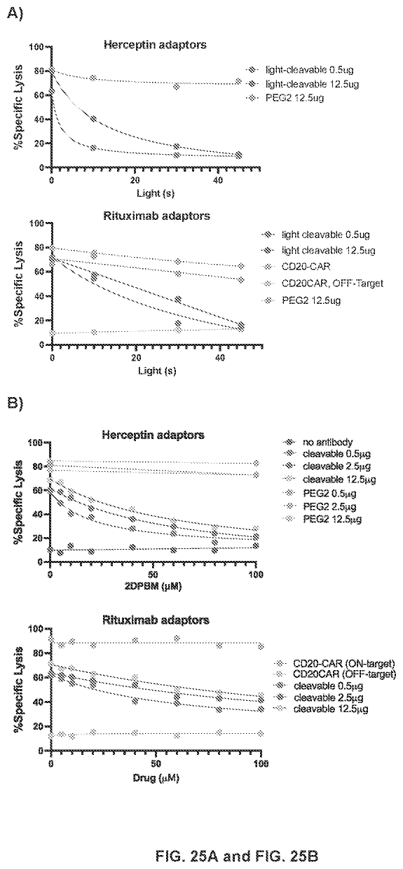

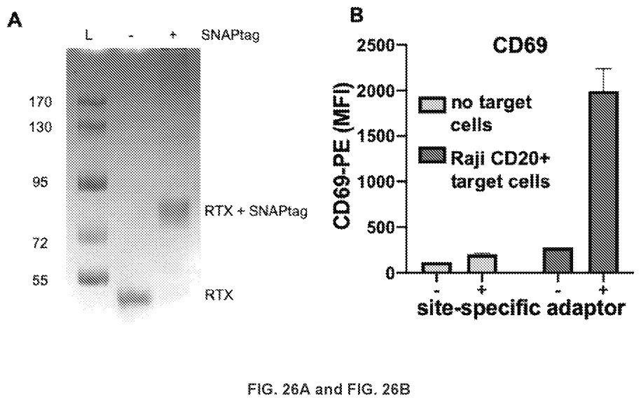

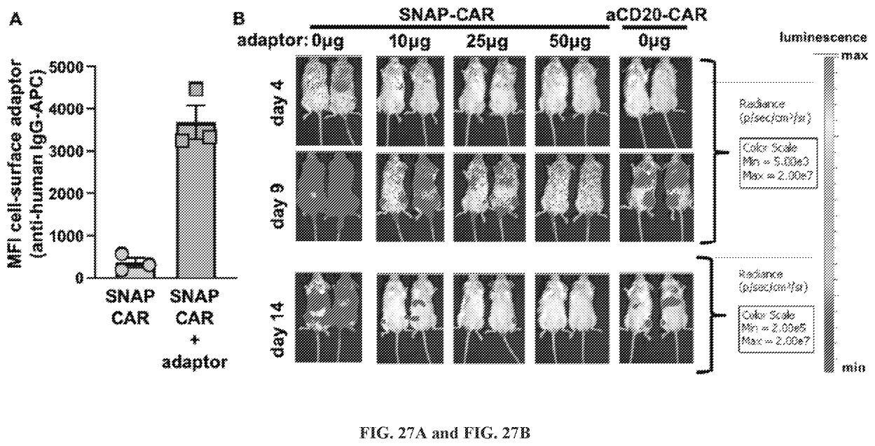

41. FIG. 13C shows alternative, red-shifted chromophores installed instead of the N3 in 16, inducing linker cleavage through a 1,6-elimination. X is the conjugating group.

42. FIGS. 14A, 14B, and 14C show clinical utility of ON-switch adaptor control. FIG. 14A shows the light-triggered ON-switch allows for external, spatial control over activity. FIG. 14B shows small molecule-triggered switch allows for tunable dosing of CAR T activation, avoiding non-specific toxicities from overactivation of CART cells. FIG. 14C shows TME-triggered switches avoid. ON-target OFF-tumor toxicities

43. FIG. 15A shows the mechanism of action for ON-switch adaptors.

44, FIG. 15B shows conditionally activated adaptors and their role in mediating CAR T cell targeting as shown in FIG. 2B are based on 3 components: conjugating group X, caging group R, and benzylguanine BG.

45. FIG. 16 shows a crystal structure of BG (teal) interacting with SNAPtag((tan). There are five residues (purple) with atoms within 4 Å of BG's exocyclic amine, indicating the ability to generate a conditionally activatable (caged) BG.

46. FIGS. 17A, 17B, and 17C show light-removable caging groups R include (17A) coumarin, (17B) BODIPY, and (17C) cyanine.

47. FIGS. 18A and 18B show the release mechanisms of caging groups R responsive to (18A) phosphine and (18B) tetrazine,

48. FIGS. 19A, 19B, 19C, and 19D show caging groups sensitive to the TME. FIG. 19A show acid and (19B) ROS sensitive caging groups and peptide structures for (19C) legumain and (19D) MMP catalyzed release of BG.

49. FIG. 20 shows the design strategy for combinatorial antigen adaptors leading to logical activation of receptor signaling and cytolysis in response to antigen combinations.

50. FIG. 21 shows proposed structures for two NTR-activated adaptors. Syntheses can follow routes developed herein, X=conjugating group.

51. FIGS. 22A and 22B show that the SNAP-synNotch receptor can be targeted to antigens of interest by BG-conjugated antibodies. FIG. 22A shows SNAP-syriNotch receptor and activation. The system triggers Gal4-VP64 transcription factor-driven TagRFP expression. FIG. 22B shows flow cytometry analysis of the activation of SNAP-synNotch cells incubated with target cell lines and indicated amounts of antibody. TagRFP levels are reported as mean fluorescence intensity (MFI), n=3 biologically independent experiments±s.d.

52. FIGS. 23A, 23B, and 23C show antibody adaptor OFF-switches that allow for stimulus-controlled display of adaptor tag molecule on the cell surface. FIG. 23A shows a diagram of a cell-surface biotin assay for measuring the accessible tag on the surface of target cells bound by adaptor OFF-switches. FIG. 23B shows cells stained with indicated amounts of OFF-switch adaptors targeting HER2 (Herceptin) or adaptor CD20 (Rituximab), and then exposed to 365 nm light for the indicated time or FIG. 23C) of the indicated amount of 2DPBM drug. Cells were incubated at 37C for 24 hrs and then stained with streptavidin-APC and assessed by flow cytometry. (Note: antibody amounts in legends correspond to staining concentrations of μg/mL)

53. FIG. 24 shows that phosphine cleavable OFF-switches can be triggered by additional phosphine drugs. Cells were stained with 0.5 μg/ml of OFF-switch adaptors targeting HER2 (Herceptin) and then exposed the indicated amount of phosphine small molecules (Bis(p-sulfonatophenyl) phenylphosphine, Tris(3 sulfonatophenyl)phosphine, 2(Diphenylphosphanyl)benzamide[2DPBM]), Cells were incubated for 2 hrs at 37C and then stained with Streptavidin-APC and assessed by flow cytometry.

54. FIG. 25A and 25B show OFF-switch adaptors mediate conditional lysis of target cells by universal CAR T cells. FIG. 25A shows mSA2 universal CAR T cells co-incubated with K562+HER2 or K562+CD20 target cells pre-stained with the indicated concentration of adaptor at a ratio E:T of 10:1), Co-cultures were then exposed to the 365 nm light for the indicated time and incubated for 24 hrs at 37C. Co-cultures were then assessed by flow cytometry for target cell lysis. FIG. 25B shows testing of the phosphine controlled OFF-switch, co-culture assays were plated identically to panel “25A” except the indicated concentration of 2DPBM drug was added to the wells and there was no light exposure. “PEG2” indicates control adaptors made with inert non-cleavable PEG linkers. CD20-CAR (ON-target) indicates anti-CD20 CAR T cells incubated with K562±CD20 target cells, and CD20-CAR (OFF-target) indicates anti-CD20 CAR T cells incubated with K562 (CD20 negative) target cells. (Note: antibody amounts in legends correspond to staining concentrations of ug/mL.)

55. FIGS. 26A and 26B show MTGase enzymatic antibody-adaptor conjugation. FIG. 26A shows that SDS-PAGE of rituximab-BG adaptor (RTX) made via MTGase conjugation, co-incubated with SNAPtag (2 eq. per BG) and visualized using Coomassie stain, reveals near-complete conjugation. FIG. 26B shows that primary human SNAP-CAR T cells that were co-incubated with MTGase-produced site-specific adaptor in the presence or absence of CD20+ Raji target cells. CD69 T cell activation marker expression on the CAR T cells was evaluated after 24 h. n=3 biol. reps.

56. FIGS. 27A and 27B show the engineered SNAP-CAR is effective in an in vivo mouse model. FIG. 27A shows NSG mice were injected intravenously (i.v.) with SNAP CAR T cells and either PBS or rituximab-BG adaptor intraperitoneally (i.p). After 24 h, blood was drawn and T cells were evaluated by flow cytometry for CAR expression and surface hound rituximab-BG adaptor (anti-human IgG-APC). n=3 mice per group. FIG. 27B shows NSG mice were challenged with 10⁶ Raji CD20+ human leukemia cells expressing firefly-luciferase followed by adaptor injection at indicated amts. on days 4 and 9 and SNAP CAR T cell injection or anti-CD20 CAR T cell injection (“aCD20”) on day 5. Raji tumor burden was evaluated by IVIS luminescence imaging at days 4, 9, and 14.

### IV. DETAILED DESCRIPTION

57. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

58. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

59. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

60. In this specification and in the claims which follow, reference will be made to a. number of terms which shall be defined to have the following meanings:

61. “Optional” or “optionally” means that the subsequently described event or circumstance may or may not occur, and that the description includes instances where said event or circumstance occurs and instances where it does not.

62. An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 65, 70, 75, 80, 85, 90, 95. or 100% increase so long as the increase is statistically significant.

63. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

64. “inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a. 10, 20, 30, 40, 50, 60, 70, 80, 90, 100%, or any amount of reduction in between as compared to native or control levels.

65. By “reduce” or other forms of the word, such as “reducing” or “reduction,” is meant lowering of an event or characteristic (e.g., tumor growth). it is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

66. By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

67. The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

68. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

69. A “control” is an alternative subject or sample used in an experiment for comparison purposes. A control can be “positive” or “negative.”

70. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Compositions

71. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular conditional universal CAR system or conditional universal SynNotch receptor is disclosed and discussed and a. number of modifications that can be made to a number of molecules including the universal CAR system or conditional universal SynNotch receptor are discussed, specifically contemplated is each and every combination and permutation of universal CAR system or conditional universal SynNotch receptor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, B-D, B-F, B-F, C-D, C-F, and C-F are considered disclosed. Likewise, any subset or combination of these is also disclosed. Thus, for example, the sub-group of A-E, B-F, and C-F would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

72. Adoptive cell therapy using antigen receptor engineered cells is a highly promising therapeutic approach, in which T cells are genetically modified to express an antigen receptor protein and are then adoptively transferred into the patient. These cells then act as a “living drug” that can elicit potent and therapeutic effects in response to sensing a target antigen on a neighboring cell anywhere in the body.

73. The most clinically advanced antigen receptor technology is chimeric antigen receptors (CARs) that consist of an antigen binding region, an extracellular spacer domain, a transmembrane domain, and T cell signaling domains (FIG. 1A). Upon antigen binding, the CARs cluster and activate the T cell receptor signaling pathway leading to lysis of target cells, cell proliferation, and production of cytokines that can amplify the immune response. CAR T cell therapy targeting the CD19 protein, an antigen found on the surface of leukemia B cells and normal B cells, is FDA-approved, showing remarkable levels of cancer remission in 85% of patients. CAR T cells targeting new antigens are being developed to treat a wide variety of diseases, including solid tumors, viral infections, and autoimmune diseases.

74. An even more versatile class of antigen receptors in development are synthetic Notch (synNotch) receptors that upon binding to a target antigen can regulate the expression of one or more therapeutic genes of interest without affecting endogenous cell signaling pathways. Modified from the Notch/Delta signaling pathway, synNotch receptors consist of an extracellular antigen binding domain, the Notch core protein, and an intracellular transcription factor. Upon antigen binding, the receptor is stretched by mechanical force, exposing an enzyme (such as a protease) cleavage site and releasing a transcription factor upon proteolysis in order to regulate gene expression (FIG. 1B). SynNotch receptors can be engineered to sense a wide range of antigens and to express a plethora of therapeutic output genes in response, including cytokines, toxins, chemokines, other receptors, and entire gene circuits. This highly programmable platform technology has been applied to various cell types, in addition to T cells, and is of great interest to the fields of immunotherapy and tissue engineering.

75. We recently created a universal adaptor CAR system as well as the first universal adaptor synNotch system using the SNAPtag-targeting domain. Universal adaptor receptors are a new generation of antigen receptors that instead of directly binding to an antigen on a target cell, bind to a common tag molecule fused or conjugated to an antigen-specific antibody, referred to as the “adaptor”. These systems are designed such that a patient is infused with an adaptor that form the physical link between the target cells and the receptor T cells (FIG. 1C). Adaptor receptors are termed “universal” as one receptor can target multiple tumor antigens within a single patient or across different disease indications simply by administering different adaptors sequentially or simultaneously. The universal SNAP receptors are unique, since instead of merely transiently interacting with the adaptor, they form a covalent bond. Through a bio-orthogonal benzylguanine (BG) tag and the SNAP receptors, we are able to target several different antigens and achieve highly potent receptor activation and signaling. The importance of adaptor CAR systems has been recognized in the development of generating adaptors based on antibodies modified with biotin, fluorescein, peptide neo-epitopes, Fey, and leucine zippers, and the first adaptor CAR system is currently in clinical trials.

76. Here, we are capitalizing on the synthetic adaptor approach to gain additional (photo)chemical or biological control over these systems, by inserting several stimulus-responsive OFF→ON and ON→OFF switches into the adaptors, that respond to inputs such as small molecules (for user-defined temporal control) and light (for user-defined temporal and spatial control), as well as pH, hydrogen peroxide, proteases and other enzymes (for highly specific activation in the tumor microenvironment; TME). The conditional ON-switch systems described here consists of adaptors that have a stimulus-reactive group, often called a ‘caging group’, blocking the tag ligand molecule on the adaptor. Without the stimulus, the caging group blocks recognition by CAR T cells, but with the stimulus, the caging group is removed allowing recognition by the CAR T cell and receptor activation. In contrast, the OFF-switches consist of adaptors with a stimulus-reactive chemical cleaving group between the tag ligand and the antibody. Thus, when no stimulus is present the adaptor is able to activate the CAR T cells, but when the stimulus is present, it will cleave off the tag ligand from the adaptor and stop it from activating the CAR T cells.

77. The unprecedented level of conditional control enabled by our light and small molecule switch systems, as well as the proposed physiological signal responsive systems allows for improved patient outcomes through lowering toxicity by improved targeting and granting the ability to treat new disease indications. While conditional control methods have been developed for CAR T cells, including activation by small molecules, light, proteases, and antigen combinations, these methods have been implemented at the level of receptor protein engineering, and require extensive genetic modifications for their implementation for every new antigen(s) targeted. By focusing engineering on the synthetic adaptor molecule within the context of the universal receptor systems, the technology provides a fundamentally new solution to the antigen receptor specificity problem, that takes advantage of technological advancements in the field of conditional linker control chemistry. This approach yields superior reagents that can all be used with the same universal cell receptor without further genetic manipulation for all modes of implementation based on the flexibility provided by the synthetic adaptors, including: 1) both ON-switches and OFF-switches, 2) several conditional control inputs (e.g., light, small molecules, etc), and 3) targeting different antigens of interest. These innovations are the first conditionally controlled universal CAR and universal synNotch receptors with improved signaling activity and flexible programmability due to covalent cell surface modification. The disclosed synNotch receptors and CAR systems achieve their universality through the use of adaptor molecules in the extracellular portion of the synNotch receptor or CAR and which adaptor molecules form a covalent bond with an antigen recognition element. This system for creating universal CARs and synNotch receptors using adaptor molecules to form a covalent bond with an antigen recognition element represents a vast improvement over existing adaptor CAR T cells and is the first successful adaptor synNotch system ever created.

78. However, this universality does not address the issue of specificity and offsite toxicities. The present disclosure solves this problem by creating conditional universal CAR systems and conditional universal SynNotch receptors. Accordingly, in one aspect, disclosed herein are conditional universal chimeric antigen receptor (CAR) system, comprising i) a CAR, comprising a receptor that targets a tag ligand on a conditional adaptor molecule, a hinge domain (such as, for example, a CD8αdomain, CD28 hinge, or modified IgG hinges with a deletion or modification of the CH2 and/or CH3 domain), and a signaling domain (such, as for example a DAP10, NKG2D, NKG2C, NKp4,4, CD28, CD27, Meg10, CD32, CD16, 2B4, or CD3δ, signaling domain); and ii) a conditional adaptor molecule comprising an antigen recognition element (such as, for example an antibody, antigen recognizing fragment thereof, a protein binding domain, lectin, DNA aptarner, RNA aptarner, a small molecule ligand for cell surface receptor, or a peptide/protein ligand for natural protein receptor), a stimulus reactive group, and a tag ligand (such as, for example, benzylguanine (BG), fluoresceine (FITC), chloroalkane, SpyTag, leucine-zipper, La-SS-B, CD19, anti-folate receptor antibody, Fc domain, peptide neoepitope (PNE), biotin, and benzylcytosine (BC)). Also disclosed herein are conditional universal synthetic Notch (synNotch) receptors comprising a conditional adaptor molecule comprising a stimulus reactive group and a tag ligand (such as, for example, benzylguanine (BG), FITC, SpyTag, leucine-zipper, La-SS-B, CD19, anti-folate receptor antibody, Fc domain, peptide neoepitope (PNE), biotin, chloroalkane, and benzylcytosine (BC)), a notch core comprising one or more cleavage sites, and one or more transcription factors.

79. Both the universal synNotch receptors and the universal CAR systems disclosed herein comprise adaptor molecules. These adaptor molecules facilitate the formation of a binding interaction with an antigen recognition element (such as for example, an antibody or antibody fragment). The binding interaction can be a covalent bond, non-covalent bond, or other interaction such as a receptor-ligand or antibody-antigen/peptide/protein binding interaction (such as, for example FITC and anti-FITC or biotin/avidin). When covalently bonded, covalent bonding can occur through pi-clamp; ligand directed tosyl chemistry; recombinant antibodies with tag ligand incorporation (such as, for example, BG or BC incorporation) through short peptide tags, sortase mediated labeling; unnatural amino acid mutagenesis followed by ‘click’ chemistry, [3+2] cycloaddition, split inteins, THIOMABs, tetrazine ligation, Staudinger ligation, imine formation, thiol-ene reaction, native chemical ligation; biotin ligase mediated labeling; lipoic acid ligase mediated labeling; NHS-ester conjugation, conjugation to cysteine, disulfide re-stapling via bis-sulfones or other reagents, glycan conjugating chemistry, or formyl glycine conversion. Covalent bond formation can also occur through the use of adaptor molecules that comprise polypeptide tags that covalently bind a target modification. Examples of polypeptide adaptor molecules include, but are not limited to SNAP-tag (which covalently bonds to a O⁶-benzylguanine which can be inserted into the antigen recognition element), CLIP-tag (which covalently bonds to a O²-benzylcytosine which can be inserted into the antigen recognition element), Halo-tag (which covalently bonds to a chloroalkane linker which can be inserted into the antigen recognition element), SpyTag (which covalently bonds to a Spy catcher peptide sequence which can be inserted into the antigen recognition element), SnoopTag (which covalently bonds to a Snoop catcher peptide sequence which can be inserted into the antigen recognition element), or Isopep-tag (which covalent bonds to its biding partner which can be inserted into the antigen recognition element). The formation of a covalent bond is a key improvement over other adaptor CAR T cell systems which rely on week interactions and which is sufficiently strong such that the bond is not broken upon antigen binding allowing for the Notch cleavage site(s) to be revealed in the synNotch receptor.

80. Also disclosed herein are conditional universal CAR systems and synNotch receptors, wherein the stimulus to which the stimulus reactive group is reactive comprises a light (including, but not limited to light in the visible light spectrum (400-650 nm), laser light source sending incident light into the patient's tissues that includes light from 650-790 nm, and light at 800-840 nm) (for example, light at a wavelength of 365, 405, 544, 780 nm)), enzyme (such as, for example, legumain, matrix metalloproteinase, pyridoxal kinase (PDXK), aldehyde dehydrogenase 7 family, member A1, (ALDH7A1), lipase C, hepatic type (LIPC), poly(ADP-ribose) polymerase 1 (PARP1), pyruvate kinase M2 (PKM2), phosphoglycerate kinase 1 (PGK1), ketohexokinase-A (KHK-A), hexokinases (HK), nucleoside diphosphate kinase (NDPK or NDK), and 6-phosphofructo-2-kinase/fructose-2,6-biphosphatase 4 (PFKFB4), mitochondrial α-ketoglutarate dehydrogenase (α-KGDH), lysines acetyltransferase 2A (KAT2A), acetyl-CoA synthetase short-chain family member 2 (ACSS2), ATP-citrate lyase (ALLY), pyruvate dehydrogenase complex (PDC), α-ketoglutarate dehydrogenase (α-KGDH), CD39, CD 73, or fumarase), small molecule (such as, for example, phosphine or tetrazine), pH, hypoxia (for use hypoxia reactive motifs such as, for example, nitroaromatics), H₂O₂, and/or reactive oxygen species (ROS) (for use ROS reactive motifs such as, for example a ROS-labile linker: N¹-(4-boronobenzyl)-N³-(4-boronophenyl)-N¹,N¹,N³,N³-tetramethylpropane-1,3-diaminium (TSPBA).

81. In one aspect, disclosed herein are conditional universal CAR systems, wherein the stimulus reactive group comprises a cleavable linker (such as, for example, a photocleavable or phosphine cleavable linker) or a stimulus reactive caging group (for example, a light reactive caging group comprising nitrobenzyl, coumarin, BODIPY, or cyanin) that blocks the CAR from binding to the tag ligand.

82. it is understood and herein contemplated that the antigen recognition element can be an antibody or any antigen recognizing fragment thereof (such as, for example, Fab, Fab′2, scFV, Fv, and the like). In one aspect, the antigen recognition element can comprise an anti-cancer-based monoclonal antibodies such as cetuximab (anti-EGFR), nimotuzumab (anti-EGFR), panitumumab (anti-EGFR), retuximab (anti-CD20), omalizumab (anti-CD20), tositumomab (anti-CD20), trastuzumab (anti-Her2), herceptin (anti-Her2), gemtuzumab (anti-CD33), alemtuzumab (anti-CD52), FMC63 (anti-CD19), and bevacuzimab (anti-VEGF) or antigen recognizing fragment thereof. In some aspects, the antigen recognition element can comprise protein binding domains (such as, for example, nanobodies and single domain antibodies (e.g., monobodies), lectins, DNA aptamers, RNA aptamers, any small molecule ligands for cell surface receptors (e.g., folic acid which is bound by the folic acid receptor), peptide/protein ligands for natural protein receptors (such as, for example, NKG2D and/or cytokines which can be bound to their natural receptors).

83. In one aspect, it is understood and herein contemplated that for T cell activation of the CAR T cell to occur additional cellular signaling events need to occur beyond the CAR T cell antigen recognition element binding to its target. Co-stimulation is also required. Co-stimulation can occur via native interactions that occur during the activation of any T cell and already present on any CAR T cell such as the stimulation of CD28 and 4-1BB via interactions with their respective ligands B7 and 4-1BBL on the surface of the target cell. Alternatively, the universal CAR can further comprise one or more co-stimulation domains (such as, for example, signaling domains for CD27, CD28, ICOS, 4-1BB, or OX40), such that co-stimulation occurs upon the antigen recognition element binding its target without the further need of the target cell providing the necessary co-stimulatory signals (FIGS. 5A and 5B). Thus, in one aspect, disclosed herein are universal CAR comprising an adaptor molecule, a hinge domain (such as, for example, a CD8α domain, CD28 hinge, or modified IgG hinges with a deletion or modification of the CH2 and/or CH3 domain), and a signaling domain (such, as for example a DAP10, NKG2D, NKG2C, NKp44, CD28, CD27, Meg10, CD32, CD16, 2B4, or CD3δ, signaling domain); and wherein the CAR further comprises one or more co-stimulation domains (such as, for example, signaling domains for CD27, CD28, ICOS, 4-1BB, or OX40), Also disclosed herein are universal CAR T cells expressing any of the universal CARs disclosed herein further comprising one or more co-stimulation domains. Thus, in one aspect, disclosed herein are conditional universal CAR systems, further comprising one or more co-stimulation domains (such as, for example, CD27, CD28, ICOS, 4-1BB, or OX40).

84. It is understood and herein contemplated that the disclosed CARs and synNotch receptors are made from and/or ultimately expressed on T cells, NK cells, NK T cells, B cells, and/or macrophage for the CAR and any immune cell (e.g., T cell, a B cell, memory T memory B cell, NK T cell, a monocyte, a natural killer cell, a dendritic cell, a macrophage, a regulatory T cell, a helper T cell, γδ T cell, or a cytotoxic T cell), a neuron, an epithelial cell, and endothelial cell, or a stem cell for the synNotch receptor. The cells used to make and express the universal CARs and synNotch receptors disclosed herein as well as any cell comprising said receptors may be from an autologous, syngeneic or allogeneic source with the selection dependent on the disease to be treated and the means available to do so. When a T cell, suitable populations of effector cells that may be used in the methods include any immune cells with cytolytic activity, such as T cells. Exemplary sub-populations of T cells include, but are not limited to those expressing CD3+ including CD3+CD8+ T cells. CD3+CD4+ T cells, and NKT cells. In one aspect, the T cells are peripheral blood mononuclear cells (PBMC) of any HLA background from PBMCs and utilized in an autologous, syngeneic, or allogeneic systems. T cells may also be isolated from any source, including but not limited to a tumor explant of the subject being treated or intratumoral T cells of the subject being treated. For the sake of convenience, the effector cells are commonly referred to herein as T cells, but it should be understood that any reference to T cells, unless otherwise indicated, is a reference to all effector cell types as defined herein. Accordingly, disclosed herein are engineered T cells comprising the universal CAR (universal CAR T cells) and/or universal synNotch (engineered universal synNotch T cell) disclosed herein. In one aspect, it is understood and herein contemplated that the synNotch receptor and CAR can be expressed on the same T cell. In such situations the antigen recognition element can be the same, allowing both synNotch transactivation of cytokines and T cell activation to occur. Alternatively, the synNotch receptor and CAR can comprise different antigen recognition elements. To ensure that the antigen recognition element on the CAR and synNotch are different, where both are present on the same cell, the synNotch receptor and CAR can comprise different adaptor molecules allowing for a different covalent interaction. Thus, in one aspect, disclosed herein are engineered T cell comprising any of the universal CAR and the universal synNotch receptors disclosed herein, wherein the CAR and synNotch receptor comprise different adaptor molecules.

85. As noted above, the effector action of the universal synNotch receptor occurs through the transcriptional activation of response genes in the cell. The one or more transcription factors (such as, for example, Gal4-VP64, Gal4-VP16, TetR-VP64, LacI-VP64, and the like) can be specifically designed to activate transcription of I cell where the response genes can be T cell effector molecules including, but not limited to IL-4, IL-10, FASL, IFN-γ, TNF-α granzyme A. granzyme B. granulysin, and/or perforin. The transcriptional activation can occur through the use of native or designer transcription factors (Crispr/Cas9, TALEN, or zinc finger). Thus, in once aspect, disclosed herein are engineered T cells comprising a universal synthetic Notch (synNotch) receptor, wherein one or more transcription factors of the universal synNotch receptor activate expression of one or more native cell response genes (such as, for example, T cell effector molecules IL-4, IL-10, FASL, IFN-γ, TNF-α, granzyme A. granzyme B, granulysin, and/or perforin). Alternatively, it is contemplated herein that the transcription factor (such as, for example, Gal4-VP64, Gal4-VP16, TetR-VP64, LacI-VP64, and the like) can be specific for a transcriptional response element (such as, for example, Gal4-VP64, Gal4-VP16, TetR-VP64, LacI-VP64, and the like) on a vector expressing transgene system allowing for unique non-native interaction and expression of one or more response genes (such as, for example, IL-4, IL-10, FASL, IFN-γ, TNF-α, granzyme A, granzyme B, granulysin, and/or perforin). One or more response genes can be encoded on the vector along with the transcriptionsal response element and a promoter which drives the expression of the effector molecule. Thus, in one aspect, disclosed herein are engineered cells comprising a universal synthetic Notch (synNotch) receptor, further comprising a vector comprising with a transcriptional response element operatively linked to a promoter driving expression of one or more response genes (such as, for example, T cell effector molecules IL-4, IL-10, FASL, IFN-γ, TNF-α, granzyme A. granzyme B, granulysin, and/or perforin); wherein the transcriptional response element is specific for one or more of the transcription factors on the synNotch receptor,

86. In some aspects, the conditional universal synNotch receptors, can further comprising an antigen recognition element (such as, for example an antibody, antigen recognizing fragment thereof, a protein binding domain, lectin, DNA aptamer, RNA aptamer, a small molecule ligand for cell surface receptor, or a peptide/protein ligand for natural protein receptor); wherein the antigen recognition element is or can become covalently linked to the conditional universal adaptor molecule. In some aspect, the antigen recognition element comprises rituximab, FMC63, herceptin, cetuximab, nimotuzumab, panitumumab, omalizumab, tositurnornab, trastuzumab, geintuzumab, alemtuzumab, bevacuzimab or an antigen-binding fragment of any none thereof.

87. Also disclosed herein are engineered cells (such as, for example, an immune cell, a neuron, an epithelial cell, and endothelial cell, or a stem cell) comprising any of the conditional, universal synNotch receptors disclosed herein. In one aspect, disclosed herein are engineered cells, further comprising a vector comprising a transcriptional response element operatively linked to a promoter driving expression of one or more cell response genes (such as, for example IL-4, IL-10, FASL, IFN-γ, TNF-α, granzyme A. granzyme B, granulysin, and/or perforin); wherein the one or more of the transcription factors on the synNotch receptor are specific for the transcriptional response element. Also disclosed herein are engineered cells, wherein one or more transcription factors of the conditional universal synNotch receptor activate expression of one or more native cell response genes (such as, for example, IL-4, IL-10, FASL, IFN-γ, TNF-α, granzyme A. granzyme B, granulysin, and/or perforin).

1. Homology/identity

88. It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

89. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Biol, 48: 443 (1970), by the search for similarity method of Pearson and Lipman, Proc. Nati. Acad. Sci. USA 85: 2444 (1988), by computerized implementations of these algorithms (GAP, BESTFIT, PASTA, and TFASTA in the Wisconsin Genetics Software Package, Genetics Computer Group, 575 Science Dr., Madison, WI), or by inspection.

90. The same types of homology can be obtained for nucleic acids by for example the algorithms disclosed in Zuker, M. Science 244:48-52, 1989, Jaeger et al. Proc. Nati. Acad. Sci. USA 86:7706-7710, 1989, Jaeger et al. Methods Enzymol. 183:281-306, 1989 which are herein incorporated by reference for at least material related to nucleic acid alignment.

2. Delivery of the Compositions to Cells

91. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods can be modified to specifically function with large DNA molecules. Further, these methods can be used to target certain diseases and cell populations by using the targeting characteristics of the carrier.

### a) Nucleic acid-based delivery systems

92. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

93. As used herein, plasmid or viral vectors are agents that transport the disclosed nucleic acids, such as chimeric antigen receptor or synNotch into the cell without degradation and include a promoter yielding expression of the gene in the cells into which it is delivered. Viral vectors are, for example, Adenovirus, Adeno-associated virus, Herpes virus, Vaccinia virus, Polio virus, neuronal trophic virus, lentiviruses, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type can carry coding regions for Interleukin 8 or 10.

94. Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

95. A retrovirus is an animal virus belonging to the virus family of Retroviridae, including any types, subfamilies, genus, or tropisms, including, but not limited to lentiviruses (including HIV based lentiviral vectors and gammaretroviral vectors). Retroviral vectors, in general, are described by Verma, I. M., Retroviral vectors for gene transfer.

96. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the IRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol. and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

97. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

98. The construction of replication-defective adenoviruses has been described (Berkner et at., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-113 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (199.3); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell, 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

99. A viral vector can be one based on an adenovirus which has had the El gene removed and these virons are generated in a cell line such as the human 293 cell line. In another preferred embodiment both the E1 and E3 genes are removed from the adenovirus genome.

### (3) Adeno-associated Viral Vectors

100. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco. CA, which can contain the herpes simplex virus thymidine kinase gene, HSV-tk, and/or a marker gene, such as the gene encoding the green fluorescent protein, GFP.

101. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

102. Typically, the AAV and B19 coding regions have been deleted, resulting in a. safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

103. The disclosed vectors thus provide DNA molecules which are capable of integration into a mammalian chromosome without substantial toxicity.

104. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

105. Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein:Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

106, Other useful systems include, for example, replicating and host-restricted non-replicating vaccinia virus vectors.

### b) Nose-nucleic Acid Based Systems

107. The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo Or in vitro.

108. Thus, the compositions can comprise vectors for example, lipids such as liposomes, such as cationic liposomes (e.g,, DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Moi. Biol. 1:95-100 (1989); Feigner et al. Proc. Natl. Acad. Sci USA 84:7413-7417 (1987); U.S. Pat. No.4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

109. In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc,, Gaithersburg, MD), SUPERFECT (Qiagen, Inc. Hilden, Germany) and TRANSFECTAM (Promega Biotec, Inc., Madison, WI), as well as other liposomes developed according to procedures standard in the art, In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

110. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Seater, et al., Bioconnigaie Chem., 2:447-451, (1991); Bagshawe, K. D., Br. J. Cancer, 60:275-281, (1989); Bagshawe, et al., Br. J. Cancer. 58:700-703. (1988) Seater, et al., Bioconjugate Chem., 4:3-9, (1993); Battelli., et al., Cancer Immunoi. Immunother., 35:421-425, (1992); Pietersz and McKenzie, Immunolog. Reviews, 129:57-80, (1992); and Roffler, et al., Biochein. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica _Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency. and ligand concentration. Molecular and cellular mechanisms of receptor-mediated endocytosis has been reviewed (Brown and Greene, DNA and Cell Biology 10:6, 399-409 (1991)).

111. Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

112. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### c) In Vivo/ex Vivo

113. As described above, the compositions can be administered in a pharmaceutically acceptable carrier and can be delivered to the subject=s cells in vivo and/or ex vivo by a variety of mechanisms well known in the art (e.g., uptake of naked I)NA, liposome fusion, intramuscular injection of DNA via a gene gun, endocytosis and the like).

114. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

3. Expression Systems

115. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

116. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et at., Nature, 273: 113 (1978)). The immediate early promoter of the human cytomegalovirus is conveniently obtained as a HindIII E restriction fragment (Greenway, P. J. et al., Gene 18: 355-360 (1982)), Of course, promoters from the host cell or related species also are useful herein.

117. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Rio, 4: 1293 (1984)). They are usually between 10 and 300 by in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

118. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

119. In certain embodiments the promoter and/or enhancer region can act as a. constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

120. It has been shown that all specific regulatory elements can be cloned and used to construct expression vectors that are selectively expressed in specific cell types such as melanoma cells. The glial fibrillary acetic protein (GTAP) promoter has been used to selectively express genes in cells of glial origin.

121. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

122. The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

123. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thytni dine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

124. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin.), xgpt (mycophenolic acid) or h.ygrotnycin, respectively. Others include the neomycin analog G418 and puramycin.

4. Antibodies

### (1) Antibodies Generally

125. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with a given antigen target. The antibodies can be tested for their desired activity using the in vitro assays described herein, or by analogous methods, after which their in vivo therapeutic and/or prophylactic activities are tested according to known clinical testing methods. There are five Major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

126. The term “monoclonal antibody” as used herein refers to an antibody obtained from a substantially homogeneous population of antibodies, the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

127. The disclosed monoclonal antibodies can be made using any procedure which produces mono clonal antibodies. For example, disclosed monoclonal antibodies can be prepared using hybridoma methods, such as those described by Kohler and. Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

128, The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

129, In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566, Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

130. As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain target binding activity are included within the meaning of the term “antibody or fragment thereof,” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

131, Also included within the meaning of “antibody or fragments thereof” are conjugates of antibody fragments and antigen binding proteins (single chain antibodies).

132. The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional propel ty, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment, (Zoller, M. J. Curr. Opin. Biotechnol. 3:348-354, 1992),

133. As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

134. The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

135, Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

136. To generate a humanized antibody, residues from one or more complementarity determining regions (CDRs) of a recipient (human) antibody molecule are replaced by residues from one or more CDR.s of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

137. Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No, 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No, 6,1.80,377 (Morgan et al.).

### 5. Aptamers

138. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,29:3), Aptamers can bind very tightly with kds from the target molecule of less than 10⁻¹²M. It is preferred that the aptamers bind the target molecule with a k_{d} less than 10⁻⁶, 10⁻⁸, 10⁻¹⁰, or 10⁻¹² . Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer has a kd with the target molecule at least 10, 100, 1000, 10,000, or 100,000 fold lower than the kd with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5.631,146, 5,731,424, 5,780,228, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

6. Pharmaceutical Carriers/Delivery of Pharmaceutical Products

139. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

140. The compositions may be administered orally, parenterally (es,, intravenously), by intramuscular injection, by intraperitoneal injection, transdermally, extracorporeally, topically or the like, including topical. intranasal administration or administration by inhalant. As used herein, “topical intranasal administration” means delivery of the compositions into the nose and nasal passages through one or both of the nares and can comprise delivery by a. spraying mechanism or droplet mechanism, or through aerosolization of the nucleic acid or vector. Administration of the compositions by inhalant can be through the nose or mouth via delivery by a spraying or droplet mechanism. De:lively can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

141, Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

142, The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bloconjugate Chem., 4:3-9, (1993); Battelli, et al., Cancer Immunol. Immunother., 35:421-425, (1992); Pietersz and. McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1990).

### a) Pharmaceutically Acceptable Carriers

143. The compositions, including antibodies, can be used therapeutically in combination with a pharmaceutically acceptable carrier.

144. Suitable carriers and their formulations are described in Remington: The Science and Practice of Pharmacy (19th ed.) ed. A. R. Gennaro, Mack Publishing Company, Easton, PA 1995, Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The p1′1 of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g,, films, liposomes or microparti cies. It will be apparent to those persons skilled in the art that certain carriers may be more preferable depending upon, for instance, the route of administration and concentration of composition being administered.

145. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

146. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinfiammatory agents, anesthetics, and the like.

147. The pharmaceutical composition may be administered in a number of ways depending on whether local or systemic treatment is desired, and on the area to be treated. Administration may be topically (including ophthalmically, vaginally, rectally, intranasally), by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

148. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

149. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

150. Compositions for oral administration include powders or granules, suspensions or solutions in water or non-aqueous media, capsules, sachets, or tablets. Thickeners, flavorings, diluents, emulsifiers, dispersing aids or binders may be desirable.

151. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid. succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

152. Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357 Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

7. Kits

153. Disclosed herein are kits that are drawn to reagents that can be used in practicing the methods disclosed herein. The kits can include any reagent or combination of reagent discussed herein or that would be understood to be required or beneficial in the practice of the disclosed methods and making the disclosed universal CAR, universal synNotch, universal CAR T cells, and/or universal synNotch cells. For example, the kits could include antibodies or fragments thereof and expression vectors to discussed in certain embodiments of the methods and composition, as well as the buffers and enzymes required.

C. Method of Treating Disease

154. The disclosed conditional universal synNotch cells and conditional universal CAR systems disclosed herein can be used to treat any disease where uncontrolled cellular proliferation occurs such as cancers, autoimmune disorders, autointlammatory disorders, and infectious disease. Accordingly, in one aspect, disclosed herein are methods of treating, decreasing, reducing, inhibiting, ameliorating, and/or preventing a cancer and/or metastasis, autoimmune disorders, autoinfla.mmatory disorders, and infectious disease in a subject comprising administering to the subject a therapeutically effective amount of any of the engineered universal CAR systems and/or engineered conditional universal synNotch cells disclosed herein.

155. “Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pall atively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection,

156. The term “treatment” refers to the medical management of a patient with the intent to cure, ameliorate, stabilize, or prevent a disease, pathological condition, or disorder, This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

157. “Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intra-ventricular, intracranial, intraperitoneal, intralesional, intranasal, rectal, vaginal, by inhalation, via an implanted reservoir, parenteral (e.g., subcutaneous, intravenous, intramuscular, intra-articular, intra.-synovial, intrasternal, intra.thecal intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

158. The term “therapeutically effective” refers to the amount of the composition used is of sufficient quantity to ameliorate one or more causes or symptoms of a disease or disorder. Such amelioration only requires a reduction or alteration, not necessarily elimination.

159. As noted above, the disclosed CAR systems and/or synNotch cells can be used to treat, decrease, reduce, inhibit, ameliorate, and/or prevent a cancer and/or metastasis in a subject. A non-limiting list of different types of cancers that can be treated through the administration of the disclosed universal CAR T cells and/or universal synNotch cells is as follows: lymphomas (Hodgkins and non-Hodgkins), leukemias, carcinomas, carcinomas of solid tissues, squamous cell carcinomas, adenocarcinoma.s, sarcomas, gliomas, high grade gliomas, blastomas, neuroblastomas, plasmacytomas, histiocytomas, melanomas, adenomas, hypoxic tumors, myelomas, AIDS-related lymphornas or sarcomas, metastatic cancers, or cancers in general.

160. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

161. Compounds disclosed herein may also be used for the treatment of precancer conditions such as cervical and anal dysplasias, other dysplasias, severe dysplasias, hyperplasias, atypical hyperplasias, and neoplasias.

162. It is intended herein that the disclosed methods of inhibiting, reducing, and/or treating a cancer can comprise the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (liniquimod), Aldesleukin, Alecensa (Alectinib), Alectinib, Alemtuzurnab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofaturaumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, 13exarotene, Bexxar (Tositumomab and. Iodine I 131 Tositumomab), Bicalutamide, BiCNU (Carmustine), Bleomycin, Blinatumomab, Blincyto (Blinaturnornab), Bortezomib, Bosulif (Bosutinib). Bosutinib, Brentuximab Vedotin, Brigatinib, BuMel, Busulfan, Busulfex (Busulfan), Cabazitaxel, Cabotnetyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil-Topical), Carboplatin, CARBOPLATIN-TAXOL, Carfilzomib, Carmubris (Carmustine), Carmustine, Carmustine Implant, Casodex (Bicalutamide), GEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobitnetinib, Cometriq (Ca.bozantinib-S-Malate), Copanlisib Hydrochloride, COPDAC, COPP, COPP-ABV, Cosmegen (Dactinomycin), Cotellic (Cobimetinib), Crizotinib, CVP, Cyclophospha.mide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, flutamide, Folex (Methotrexate), Folex PFS (Methotrexate), FOLFIRI, FOLFIRI-BEVACIZIJMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), tIemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVRD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamiduin (Ifosfamide), IL-2 (Aldesleukin), hnatinib Mesylate, Imbruvica (Ibrutinib), Imfinzi (Durvalutnab), Imiquimod, hnlygic (Talimogene Laherparepvec), Inlyta (Axitinib), inotuzurnab Ozogamicin, Interferon Alfa-2b, Recombinant, Interleukin-2 (Aldesleukin), Intron A (Recombinant Interferon Alfa-21), Iodine I 131 Tositumomab and Tositumomab, Ipilimumab, Iressa (Gefitinib), frinotecan Hydrochloride, irinotecan Hydrochloride Liposome, Istodax (Rornidepsin), Ixabepilone, Ixazornib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisgali (Ribociclib), Kytmiah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Margibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna.), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride) Mutamycin (Mitomycin C), Myleran (Busulfan), Mylosar (Azacitidine), Mylotarg (Gemtuzumab Ozogamicin), Nanoparticle Paclitaxel (Paditaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib. Nilutamide, Ninlaro (Ixazomib Citrate), Niraparib Tosylate Monohydrate, Nivolumab, Nolvadex (Tamoxifen Citrate), Nplate (Romiplostitn), Obinutuzumab, Odonizo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Pefjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methyl naltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Cam sylate, Ruxolitinib Phosphate, Rydapt (Midostaurin), Sclerosol intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafiniar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna. (Nilotinib), Taxol (Paclitaxel), Taxotere (Docetaxel), Tecentriq, (Atezolizumab), Temodar (Temozolomide), Temozolomide, Temsirolimus, Thalidomide, Thalornid (Thalidomide), Thioguanine, Thiotepa, Tisagenlecleucel, Tolak (Fluorouracil-Topical), Topotecan Hydrochloride, Toremifene, Torisel (Temsiroiimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vint)Lasalle Sulfate), Vemurafenib, Venclexta (Venetoclax), Venetoclax, Verzenio (Abentaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Vottient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy Yondelis (Trabectedin), Zaltrap (Ziv-Aflibercept), Zarxio (Filgrastim), Zejula (Niraparib Tosylate Monohydrate), Zelboraf (Vernurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza. (Vorinostat), Zometa (Zoledronic Acid), Zydelig Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab).

163. As noted above, the disclosed conditional universal synNotch cell and/or conditional universal CAR systems can be used to treat autoimmune diseases (i.e., a set of diseases, disorders, or conditions resulting from an adaptive immune response (T cell and/or B cell response) against the host organism). Examples of autoimmune diseases including, but not limited to Achalasia, Acute disseminated encephalomyelitis, Acute motor axonal neuropathy, Addison's disease, Adiposis dolorosa Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia., Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polvendocrine syndrome, Autoimmune retinopathy, Autoimmune urticaria, Axonal & neuronal neuropathy (ASIAN), Baló disease, Behcet's disease, Benign mucosal emphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophi lie Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type I, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinernia, IgA Nephropathy, IgG4-related sclerosing disease, Immune thrombocytopenic purpura (ITP), Inclusion body myositis (IBM), Interstitial cystitis (IC), Inflarnatoty Bowel Disease (IBD), Juvenile arthritis, Juvenile diabetes (Type I diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCHD), Urticaria, Urticarial vasculitis, Uveitis, Vasculitis, Vogt-Koyanagi-Harada Disease, and Wegener's granulomatosis (or Granulomatosis with Polyangiitis (GPA)).

164. Also disclosed herein are methods of treating an autoinflammatoty diseases (i.e., disorders where the innate immune response attacks host cells) comprising administering to a subject with an autoinflammatory disease the universal CAR T cells and/or universal synNotch cells disclosed herein. Examples of autoinflammatory disorders include asthma, graft versus host disease, allergy, transplant rejection, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)—Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1β (IL-1β) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behcets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID).

165. As noted above, the disclosed conditional universal CAR systems and conditional universal synNotch cells can be used to treat disease resulting from an infection with a bacterium, virus, fungi, and/or parasite.

166, In one aspect, the infectious disease being treated can be the result of an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus. Hepatitis A virus, Hepatitis B virus, Hepatitis C virus, Hepatitis D virus, Hepatitis E virus, Rhinovirus, Coronavirus (such as, for example, avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV) (SARS-CoV), Severe Acute Respiratory Syndrome (SARS)-Coronavirus (CoV)-2 (SARS-CoV-2) (including, but not limited to the B1.351 variant, B.1.1.7 variant, USA-WA1/2020, or P.1 variant), or middle east respiratory syndrome (MERS) coronavirus (CoV) (MERS-CoV)), Influenza virus A, Influenza virus B, Measles virus, Polyomavirus, Human Papilomavirus, Respiratory syncytial virus, Adenovirus, Coxsackie virus. Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus. Simian Immunodeficiency virus, Human Immunodeficiency virus type-1, and Human immunodeficiency virus type-2.

167. In one aspect, the infectious disease being treated can be the result of an infection with a bacteria selected from the group of bacteria consisting of Mycobaterium tuberculosis, Mycobaterium bovis, Mycobaterium bovis strain BCG, BCG substrains, Mycobaterium avium, Mycobaterium intracellular, Mycobaterium africanum, Mycobaterium kansasii, Mycobaterium marinum, Mycobaterium ulcerans, Mycobaterium aviuin subspecies paratuberculosis, Nocardia asteroides, other Nocardia species, Legionella pneumophila, other Legionella species, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholdeda psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibtio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In one aspect the bacteria is not Bacillus anthracis.

168. In another aspect, the infectious disease being treated can be the result of an infection with a fungi selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carnii, Penicillium marneffi, and Alternatia alternata.

169. In another aspect, the infectious disease being treated can be the result of an infection with a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowled, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Cionorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

D. Regenerative Medicine

170. It is understood and herein contemplated that disclosed conditional universal SynNotch receptor has uses not limited to the treatment of cancer, autoimmune disease, autoinflammatory disease, or infectious disease. In one aspect, the disclosed conditional universal SynNotch can be used in cellular and tissue engineering efforts to restore or establish normal cellular, tissue, and/or organ function (i.e., regenerative medicine). As noted above, disclosed herein, in one aspect, are engineered cells (such as, for example, an immune cell, a neuron, an epithelial cell, and endothelial cell, or a stem cell) comprising any of the conditional, universal synNotch receptors disclosed herein. In one aspect, disclosed herein are engineered cells, further comprising a vector comprising a transcriptional response element operatively linked to a promoter driving expression of one or more cell response genes (such as, for example IL-4, IL-10, FASL, IFN-γ, TNF-α, granzyme A. granzyme B, gra.nulysin, and/or perforin); wherein the one or more of the transcription factors on the synNotch receptor are specific for the transcriptional response element. Also disclosed herein are engineered cells, wherein one or more transcription factors of the conditional universal synNotch receptor activate expression of one or more native cell response genes (such as, for example, IL-4, IL-10, FASL, IFN-γ, TNF-α, granzyme A. granzyme B, granulysin, and/or perform). In one aspect, also disclosed herein are the use of said engineered cells to restore or establish normal cellular, tissue, and/or organ function.

### E. Examples

171. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for, Unless indicated otherwise, parts are parts by weight, temperature is in ° C. or is at ambient temperature, and pressure is at or near atmospheric.

1. Example 1: SNAP CAR T Cells and SNAP SNAP SynNotch Receptors

172. We recently developed SNAP CAR and SNAP synNotch universal receptors capable of being post-translationally targeted with BG-conjugated antibody adaptors to multiple antigens of interest (FIG. 3). These versatile receptors form a covalent bond with the antibody allowing for highly potent re-targeting of receptor activity at low concentrations of antibody adaptor. Development of the SNAP receptors directly built onto work creating a high affinity biotin-binding receptor CAR, using the monomeric streptavidin2 protein (m SA2) that is activated by biotinylated antibodies bound to their target antigens. Potent activity with the higher affinity of mSA2 compared to a previously biotin-binding CAR led to reasoning that an even higher affinity interaction, specifically a covalent bond, provides superior outcomes. Indeed, the SNAP receptors showed greatly enhanced levels of T cell-induced lysis and signaling. Moreover, we achieved a functional SNAP-synNotch system, while mSA2 was ineffective, further supporting the importance of covalent attachment.

### a) Adaptor Targeting of SNAP CAR T Cell Activation

173. We constructed stably transduced primary human T cells the universal SNAP-CAR that activate cell signaling upon labeling with BG-conjugated adaptor antibodies on the surface of target cells (FIG. 4A). To generate the adaptor antibodies, we conjugated a BG-N-hydroxysuccinimidyl (NHS) ester to lysines and/or N-termini of several clinically-relevant antibodies, including rituximab, FMC63, herceptin, and cetuximab. We determined an average of 2 BG motifs per antibody from the labeling reaction, with no positional control.

174. To test CAR functionality, we co-incubated SNAP-CAR T cells with various antigen positive or negative target tumor cell lines and BG-antibody adaptors for 24 hours, and found that the antibody-BG adaptors induced SNAP-CAR T cell activity (including T cell activation markers), interferon gamma cytokine production, as well as CAR cell-specific lysis of antigen-positive target cells (FIG. 4B,C,D). These outputs were dependent on the correct antibody-target cell combinations, demonstrating the ability to re-program T cell specificity. Importantly, it was also discovered that SNAP CAR cells can simultaneously target multiple antigens on different cell populations, which can be used to prevent cancer relapse due to antigen heterogeneity or antigen loss, in experiments co-culturing SNAP CAR T cells with a mixture of CD20+ and EGFR+target cells and assaying for cell lysis with single anti-CD20 or anti-EGFR antibodies or their combination.

### b) Function of SNAP-CAR Cells in an Animal Model

175. To demonstrate the therapeutic feasibility of using SNAP-CART cells in vivo, we carried out experiments to confirm SNAP-receptor covalent attachment to antibody adaptors in mice and evaluated anti-tumor function. We injected adaptor and SNAP CAR T cells by different routes (intraperitoneal adaptor and intravenous CART' cells) into NOD-SCID-g-chain-deficient (NSG) immunocompromised mice capable of engraftment with human cells. A day later we analyzed SNAP CAR T cells in the blood of mice and observed efficient chemical labeling of SNAP-CAR T cells by the adaptor (FIG. 28A). We then carried out a first human tumor xenograft model experiment to investigate tumor regression, and at day 14 we observed marked regression or delay in tumor growth in mice that received our SNAPCAR T cells and adaptor compared to SNAP-CAR T cells with no adaptor, similar or better than the anti-CD20 CAR positive control (FIG. 28B).

### c) Adaptor Targeting of SNAP SynNotch Receptor Activation

176. We created a universal adaptor synNotch to direct receptor activation and the resulting transcriptional gene output in response to interaction with BG-conjugated adaptor antibodies on the surface of target cells (FIG. 22). We cloned a SNAP-synNotch receptor consisting of the SNAPtag domain fused to the Notch core protein, and the Gal4-transcription factor (to turn on a gal4 promoter-driven TagBFP gene) and transduced Jurkat cells. Next, we co-incubated these cells together with the BG-conjugated anti-CD19 antibody with CD19-(+) or (−) tumor cells and observed significant up-regulation of TagBFP to CD19(+) target cells. Receptor activation was sensitive and tunable, with a significant response at an antibody concentration as low as 0.04 μglmL and increasing to a peak at 0.25 μg/mL. Response gene activation then decreased with increasing antibody indicative of a “hook effect”—as expected in ternary complex formation. BG-conjugated antibodies targeting other antigens were also capable of activating the SNAP-synNotch receptor in an antigen-specific manner indicating universality of the cell re-programming approach. We also demonstrated modularity of the SNAP-synNotch output as it was capable expressing of the 1L-7 response gene, a candidate therapeutic gene of interest for its ability to promote T cell proliferation.

### d) Light- and Small Molecule-cleavable OFF-switch Adaptors

177. To create a prototype OFF-switch adaptor (see FIG. 2), we conjugated a photo-cleavable biotin 1 to an anti-CD20 antibody via NHS ester chemistry, forming carbamates with amino groups on the protein (FIG. 5). The assembled adaptor was fully active, and 365 nm exposure of just 30 sec inactivated mSA2 CAR T cell signaling in co-cultures with antigen(+) tumor cells, as assessed by CD69 and CD62L activation marker expression.

178. In order to demonstrate small molecule controlled OFF-switching, we developed a nine-step synthesis of the phosphine-cleavable biotin NHS ester 2 containing an arylazide linker (FIG. 6A). The linker 2 was then conjugated to an anti-CD20 antibody and to test the ability of the adaptor to function on target cells, we co-incubated CD20+ cells with varying amounts of OFF-switch antibody and the small molecule trigger 2DPBM ((di phenyl phosphino)-2-benzamide). 2DPBM was selected based on previous studies using phosphines as small molecule triggers. We observed robust activation of primary mSA2 CAR T cells in the presence of the adaptor and CD20+ target cells and, importantly, inhibition of this signal when 2DPBM was added (FIG. 6B). This demonstrates conditional OFF switching of adaptor CAR activity in response to light and small molecule treatment.

179, Cleaving of the biotin motif was tested with. CD20+ target cells in response to 365 nm light titration and staining with fluorescently labeled streptavidin (FIG. 7C). In the absence of light exposure, the biotin adaptor was bound to streptavidin. Moreover, just a brief 30 s exposure was able to filly deactivate the adaptor. We also observed a robust decrease in biotin staining dependent on and correlated with small molecule drug and antibody concentrations, demonstrating tunability of cell labeling (FIG. 8C). This demonstrates conditional OFF switching of adaptor CAR activity in response to light and small molecule treatment.

### e) Light- and Small Molecule-activated ON-switch Adaptor

180. To create a prototype for the light triggered ON-switch adaptor (see FIG. 2), we synthesized the light-activated biotin sulfo-NHS ester 3 (FIG. 7A). We prepared the caging group in one step from 4,5-(methytenedioxy)-2-nitrobenzaldehyde in 86% yield, followed by assembly of 1 in three steps averaging a 63% yield. We incorporated the sulfonate to increase water solubility for the subsequent conjugation to the anti-CD20 antibody. Decaging of the biotin motif was tested with CD20+ target cells in response to 365 nm light titration and staining with fluorescently labeled streptavidin (FIG. 7B). In the absence of light exposure, the caged biotin adaptor was unable to bind to streptavidin and flow cytometry results were indistinguishable from a no-adaptor antibody control. Moreover, just a brief 30 s exposure was able to fully activate the adaptor.

181. We constructed a small molecule-triggered ON-switch again based on a Staudinger reduction and thus synthesized the arylazide caged biotin NHS ester 4 (FIG. 8A). We prepared the caging group in two steps from 4′-aminoacetophenone with a 93% average yield and then synthesized the phosphine-activated biotin NHS ester 4 in three steps. Although only the ortho isomer is shown, the para isomer was synthesized as well. Interestingly, when incubated in PBS pH17.4 at r.t,, the carbamate of the para compound completely hydrolyzed within 24 h while the ortho analogue remained stable (>90%) for one week and was therefore selected.

182. We then conjugated the stable caged biotin 3 to the anti-CD20 antibody and tested its response to 2DPBM. We observed a robust increase in biotin staining dependent on and correlated with small molecule drug and antibody concentrations, demonstrating tunability of cell labeling (FIG. 8B). This demonstrates conditional ON switching of adaptor activity in response to light and small molecule treatment.

2. Example 2: Conditional Control of Universal Antigen Receptor Signaling With OFF-switch Adaptors

183. We developed a general method for the creation of adaptor safety switches that can turn off CAR or synNotch receptor signaling using small molecules or light, thus allowing for temporal and localized toxicity mitigation from unwanted receptor activation. Light is a dynamic control stimulus that can be restricted to targeted tissues by several methods including fiberoptic probes in standard endoscopic procedures, fluorescence-based systems (used in image-guided tumor resection), and irradiation of circulating blood (not affecting organ sites). Light-controlled OFF-switches can be implemented to protect sensitive anatomical sites known to be positive for the target antigen via light exposure to these regions, while distal metastatic lesions are still being treated. HER2 and EGFR are examples of tumor antigens that are also expressed in various normal epithelial subsets and are candidates for OFF-tumor/ON-target toxicity that has led to serious toxicities in CAR T cells. Small molecule-triggered OFF-switches have complementary advantages of being routine to administer and rapidly enable complete systemic receptor deactivation, including tissue sites that cannot be reached with light probes, offering additional safety-switch features (FIG. 9). While the universal adaptor format enables some control over CAR activity through antibody dosing, the average IgG antibody half-life is 10-21 days, and while different antibody engineering approaches can shorten or lengthen this time, they still do not allow for rapid cessation required to mitigate toxicities which can occur as quickly as 15-30 minutes post-infusion. Unlike the most clinically-advanced control approach for CAR T cell therapy, a cell suicide switch, OFF-switch adaptors can allow for reversible control, without eliminating the engineered cells for potential reactivation by dosing additional—and possibly different—adaptor antibodies. SNAP receptor and antibody conjugates were found to be recycled from the cell surface and able to covalently attach to new adaptors over a petiod of 1-2 days, even more rapidly following receptor activation. The adaptor OFF-switches are building on light-cleavable and phosphine-cleavable linkers and is supported by the results on biotin OFF-switch adaptors for the mSA2-CAR. In addition to OFF-switches, we can test two different approaches for the generation of site-specific antibody conjugates with the goal of creating homogenous adaptors for predictable reagent behavior and that are suitable for structure activity relationship (SAR) studies. Homogeneous adaptors can facilitate optimization of the approaches and provide a path toward the long-term goal of clinically testing the conditionally controlled universal antigen receptor methodology.

### a) Synthesis and Validation of Site-specific Adaptor Conjugates

184. Thus far, the adaptor antibodies have been generated through reaction of protein-surface lysines, leading to heterogenous mixtures of antibody conjugates that vary with regard to number and position of the modification. As the synapse distance between the CAR T cell and target cell has been shown to be a key factor in both CAR and synNotch receptor signaling strength, it is likely that heterogenous adaptors are leading to suboptimal receptor activation. We can create site-specific installation of the adaptor molecules using rigorously tested chemistries described below, and test BG's conjugated to the enzyme via PEG spacer arms of different lengths including 2, 4, or 6 PEGs seeking to optimize the spacer length for each antibody/antigen, based on the results. We can evaluate each approach with 4 clinically approved antibodies targeting the antigen CD19, CD20, EGFR, and HER2. The synthesis of these reagents and their implementation is discussed below. Following conjugation to each antibody, antibody conjugates can be validated by mass spectrometry and purified using a buffer exchange column. Conjugation yields of bis-sulfones average 30% while those of the more recent dibromopyra.dizinediones are typically quantitative. The selected conjugation approaches allow for the use of commercial, FDA-approved antibodies with no need to engineer new sites for conjugation, thereby eliminating potential ma.nufacturinglscalability issues.

185. In the disulfide re-bridging conjugation, the antibody undergoes bis-alkylation to conjugate both thiols derived from the two cysteine residues of the reduced native disulfide bond. These reagents can undergo reaction at each of the 4 interchain disulfides of the antibody, yielding four conjugates. The product of this conjugation is stable in serum, retains antibody structure, and has been used in antibody-drug conjugates (ADCs).

186. We already prepared 5 as a common intermediate to attach different linkers for the site-specific antibody conjugation. Compound 6 can be generated through amide coupling of a PEG chain, for cell-cell synapse tuning, to a bis-sulforie-NEIS ester, and, after acid mediated deprotecti on, can be coupled to 5, delivering 7 (FIG. 10A). Additionally, we can synthesize the known dibromopyradizinedione intermediate 8 from methyl hydrazine in 4 steps and couple it to in two steps yielding 9 (FIG. 10B). These reagents enable reproducible, site-selective conjugation to antibody disulfides (FIG. 11).

187. In a complementary, enzymatic conjugation approach we synthesized the terminal amine 11 from the commercially available PEG 10 (FIG. 12A), and target a conserved glutamine located in each of the heavy chains of the de-glycosylated IgG1 antibody through selective modification by the enzyme MTGase (FIG. 12B). This generates uniform conjugates in quantitative yields adding exactly two modifications using conserved glutamines as the only γ-carbonyl amide donors on dei-glycosylated IgGls, This method that has been used for dozens of clinical use ADCs. This process generates uniform conjugates with exactly two modifications, and the method that has been used for dozens of clinical ADCs. We have now demonstrated the feasibility of this approach by generating a PEG2-BG labeled Rituximab (RTX-BG) adaptor, showing near complete BG labeling by incubation with SNAP protein (FIG. 26A). In addition, the site-specific adaptor was efficient at inducing T cell activity (FIG. 26B).

188. To evaluate the assembled adaptor-antibody conjugates for optimal activation, we can test them in receptor activation assays with SNAP-CAR and SNAP svnNotch T cells using co-incubation methodology described herein, including target lysis, T cell activation, and cytokine production (SNAP-CAR) and TagBFP production (SNAP-synNotch), Selected site-specific chemistries, based on optimal receptor activation and matched for antibody/antigen pairs, can be used to create OFF- and ON-switches in subsequent aims. One or more site-specific BG incorporation approaches leads to enhanced receptor activation compared to current heterogenous adaptors. It is possible to identify one optimal conjugation strategy, or that the optimal strategy can be dependent on the adaptor and antigen due to differences in synapse length.

### b) Synthesis and Validation of Cleavable Linker OFF-switch Adaptor Conjugates

189. Encouraged by the results for creating biotin-adaptor OFF-switches (FIGS. 5 & 6), we can carry out analogous syntheses with BG replacing the biotin motif and by applying the site-specific conjugation chemistries determined herein. We can synthesize a phosphine (Staudinger reduction)-cleavable BG via an aryl azide linker (FIG. 13A) and a light-cleavable BG via use of a caged aniline linker (FIG. 13B). Staudinger ligations have been reported in vivo and related applications of azides and arylphosphines have shown to be non-toxic and orthogonal in cells and animals. In case the Staudinger reduction cleaves BG too slowly (<90% within 4 h), we can synthesize a trans-cyclooctene linker capable of fragmenting when reacted with a tetrazine through a fast (t½<5 min) inverse electron demand Diels-Alder (IEDDA) reaction (following chemistry shown in FIG. 18).

190. To synthesize the former, 12 can be hydrolyzed under basic conditions and then coupled to an amine-bearing antibody conjugating group (e.g., a bis-sulfone) to form the amide 13. The alcohol can be activated and coupled to 5 to generate the phosphine-cleavable BG 14. To synthesize a light cleavable BG, the known intermediate 15 can be coupled to a PEG spacer to generate intermediate 16. The alcohol can be activated and coupled to the amine on 5 to form the 365 nm light-cleavable BG 17. Additionally, nicrobenzyl, coumarin, BODIPY, and cyanine-based photolabile linkers can also be synthesized. These chromophores can be cleaved at longer wavelengths (405-690 nm, FIG. 13C), reducing potential phototoxicity and enhancing tissue penetration. Following conjugation to each antibody (anti-CD19, anti-CD20, anti-EGFR, anti-HER2), conjugates can be validated by mass spectrometry and purified via buffer exchange column, as standard for ADCs.

### c) Analysis of OFF-switch Control of SNAP-CAR and SynNotch Receptor Signaling

191. We can first evaluate the OFF-switch adaptors for their ability to conditionally react with the SNAP-receptor, and then for their ability to modulate universal SNAP-CAR and SNAP-synNotch receptor signaling using co-incubation methods. For the small molecule induced OFF-switch 1614 we can apply varying amounts of 2DPBM (0-100 μM), and for the light-cleavable OFF-switch 17 varying exposure times to light (0, 30 s, 60 s, 120 s) at chromophore-matching wavelengths. Cells can then be stained with a fluorescently labeled secondary antibody that recognizes the antibody constant region of the OFF-switch adaptor. Intact adaptors are expected to be retained on the surface of SNAP receptor cells, while cleaved antibodies lacking BG will not bind. PEG-BG antibody conjugates. Foil owing confirmation of BG cleavage, deactivation of receptor signaling can be confirmed after addition of relevant the trigger stimuli.

192. Spatio-temporal control of toxicity can be directly tested using the light-triggered OFF-switch adaptors, Specifically, we can assay for the ability of light to protect a population of antigen(+) cells (mimicking antigen(+), normal cells) from killing by primary human SNAP-CAR T cells or by SNAP-synNotch cells. For synNotch cells, the TagBFP response gene can be replaced with cell-surface bound TRAIL shown to lead to target cell killing. We can monitor cell killing in real-time by fluorescence microscopy, quantifying target cell death in the light-exposed region vs. unexposed. We can stain adherent target cells SKOV-3 (HER2+ and EGFR+) cells with a caspase-activated dye (CellEvent) to monitor apoptosis. We can then label cells with OFF-switch adaptors and expose a defined region of cells to light and add SNAP receptor cells, expecting light to efficiently cleave the adaptors and inhibit target cell killing. We can also test the kinetics at which small molecules can switch OFF adaptor CAR signaling performing a time course assaying for calcium signaling by fluorescence microscopy and staining with Fluo-4AM dye. Calcium signaling is one of the earliest and fastest acting signals in CAR T cell activation. Outside the scope of this proposal, functional reagents that we are creating can undergo pre-clinical development in murine disease models. We are currently conducting animal studies for the constitutively active SNAP-CAR T cell system to develop the corresponding protocols for these long-term goals.

193. We wanted to determine if the antibody off switches allow for stimulus-controlled display of adaptor tag molecule on the cell surface. Using a cell surface biotin assay, we were able to measure the accessible tag on the surface of target cells bound by adaptor OFF-switches (FIG. 23A). We examined cells using OFF-switch adaptors targeting HER2 (Herceptin) or adaptor CD20 (Rituximab), and then exposed to 365 nm light for the indicated time (FIG. 23B). We also looked at the effect following exposure to 2DPBM. We also showed the effects observed with 2DPBM were not unique seeing a similar effect using phosphine small molecules (Bis(p-sulfonatophenyl)phenylphosphine, Tris(3-sulfonatophenyl)phosphine, 2(Diphenylphosphanyl)benzamide[2DPBM]).

194. Next we examined the ability fo the OFF-swtich adaptors to mediate conditional lysis of target cells by universal CART cells. FIG. 25A shows mSA2 universal CAR T cells were co-incubated with K562+HER2 or K562+CD20 target cells pre-stained with the indicated. concentration of adaptor and exposed to light and assessed for lysis by flow cytometry (FIG. 25A). 25B shows the same experiments without light exposure, but in the presence of 2DPBM.

3. Example 3: Conditional Control of Universal Antigen Receptor Signaling Using ON-switch Adaptors

195. We are developing universal adaptor ON-switches that, in addition to antigen sensing, require a second input trigger to activate CAR and synNotch receptor signaling (see FIG. 2B). Small molecule- and light-controlled ON-switches can provide spatiotemporal control to receptor signaling, complementary to the conditional OFF-switches generated herein allowing for controlled dose escalation and monitoring of toxicities by the clinician, either systemically with a small molecule or site-specifically using light (FIG. 14A,B). ON-switches triggered by TME stimuli can introduce an additional autonomous activation filter, yielding greater disease specificity to target antigens that are disease-associated but not disease-specific (the majority of cancer antigens), eliminating ON-target/OFF-disease toxicity (FIG. 14C).

196. The TME of solid tumors is characterized by abnormal features that also contribute to cancer progression and are shared among other disease indications. Acidosis (intra- and extracellular pH of 6.0-7.2) commonly occurs due to excess glycolysis by the tumor and hypoxia leading to increased lactic acid production, amplifying tumor growth and metastatic potential. An increase in secreted proteases such as matrix metalloprotease (MMP-2, -9, and -14) by tumor cells and or secreted by tumor-associated cells(ex: legumain), contribute to TME remodeling, cancer cell growth, metastasis, and cell survival. Hydrogen peroxide and other reactive oxygen species (ROS) are also found at elevated levels in tumor cells and extracellular space due to up-regulation of superoxide dismutase (SOD) increasing the rate of H₂O₂ production (up to 0.5 nmol/10⁴ cells/h). These triggers have already found clinical applications in pro-drugs and. ADCs.

### a) Synthesis and Validation of ON-switch Adaptors

197. Building off of the results, we can generate self-labeling enzyme substrates that can be conditionally activated using exogenous triggers or conditions specific to the tumor microenvironment. To achieve this, we can synthesize molecules bearing three components: 1) a site-specific antibody conjugating group X (e.g., bis-sulfone, or dibromopyradi zinc), 2) the SNAPtag substrate BG, and 3) the stimulus-responsive caging group R (FIG. 15B). Optimal PEGs and conjugating groups are identified herein. To cage the SNAPtag substrate and thereby —temporarily—block its interaction with the SNAPtag protein, we chose to modify the exocyclic 2-amino group on BG with carbamates and amides. Crystal structure analysis of the BG-SNAPtag interaction shows that there are five residues within a 4 Å vicinity of the 4-amino (FIG. 16). Thus, a caging group can sterically prohibit BG from fitting into the SNAPtag active site.

198. We can synthesize caging groups that can be removed using exogenous triggers or by conditions imposed by the tumor microenvironment. For light-activated SNAPtag substrates, coumarin (FIG. 17A), BODIPY (FIG. 17B), and Cyanine (FIG. 17C) based caging groups can be utilized to enable activation at various wavelengths. Two small-molecule cleavable moieties can be utilized. The phosphine triggered caging group (FIG. 18A) can be synthesized in a similar manner as in the resuits using the Sandmeyer reaction as the key step. An additional small molecule triggered caging group, TCO (FIG. 18B), is commercially available and can cleave BG when reacted with an optimized tetrazine through an inverse demand Diels Alder reaction. A hydrazone-based caging group (FIG. 19A) designed to release BG in the acidic tumor microenvironment can be synthesized with the imine formation as a key step. Aryl boronic esters (FIG. 19B) are commercially available and are reduced to alcohols in the presence of peroxides ROS in the tumor microenvironment. Additionally, caging groups sensitive to legumain (FIG. 19C) and matrix metalloproteinases (MMPs)) (FIG. 19D), can be generated via solid phase peptide synthesis. After MIP cleavage, additional cell surface proteases cleave the remaining peptide to avoid leaving a peptidic scar on the adaptor.

### b) Analysis of ON-switch Adaptor Control of SNAP-CAR and SynNotch Signaling

199. We can test anti-EGFR and anti-HER2 ON-switch adaptors for their ability to modulate universal SNAP-CAR and SNAP-synNotch receptor signaling using methodologies outlined herein in which decaging can be assessed by staining of SNAP-receptor cells in response to triggers and activation of CAR T cell and synNotch signaling in response to triggers and antigen (+) target cells. In addition to light and small molecule triggers, we can test TME stimuli including, adding recombinant proteases (MMP-2, MMP-9, MMP-14, and legumain), exogenous H₂O₂, and lactic acid (lowering media pH to 6.0). We expect triggers to efficiently decage BG moieties on the adaptors leading to high levels of SNAP staining and receptor activation with antigen-matched target cells. Spatiotemporal guidance of receptor signaling can be tested for the light-triggered ON-switch adaptors using the microscopy assays for light control established herein. We can also assay for tunable control by the 2DPBM small molecule (0-100 μM). We expect the light ON-switch adaptors to mediate potent tumor cell lysis by CAR T cells and TRAIL-producing synNotch cells only in specific wavelength light exposed regions, and for cell killing to correlate with 2DPBM dose for small molecule adaptors. Tumor microenvironment-gated control of CAR T cells can be evaluated for each TME ON-switch using established TME cell co-culture systems that mimic naturally occurring conditions and antigen(+) and (−) target cells (HER2 and EGFR), primary human SNAP CAR T cells, and ON-switch adaptors. For all co-cultures we can evaluate tumor cell killing and I cell activation by flow cytometry and cytokine production by ELISA. MMP-triggered adaptors can be tested on SKOV3 cells that naturally secrete MMPs 2,9,14 and express high levels of EGFR and HER2 antigens. Commercially available selective MMP inhibitors can be used to demonstrate the MMP-specific activity for each adaptor. For the legumain protease we can perform co-culture assays again using SKOV-3 target cells which are negative for legumain secretion but dosing in varying numbers of M2 THP-1 macrophage cells following established methods, to mimic tumor-associated macrophage production of legumain. For modeling acidosis, we can modulate pH (decreasing it) by titrating in different levels of glucose and culturing CACO-2 colon cancer cells (EGFR(+) and HER2(+)) in controlled media as described. For H₂O₂, we can again use the SKOV-3 cell line that naturally produces high levels of H₂O₂, and can demonstrate the dependency on H202 by spiking in recombinant catalase used by normal cells to remove ROS. Remodeling the TME using synNotch cells, Low level IMF infiltration by endogenous I cells or engineered T cells (CAR or TCR transgenic), is a major negative prognostic factor for cancer outcome, We can remodel the TME by increasing the T cell infiltrate by combining TME ON-switch adaptors with SNAP-synNotch cells engineered to secrete CXCL9 and CXCL10 chemokines known to selectively recruit cytotoxic T cells. We can assay for T cell recruitment by trans-well migration assays in which the supernatant from synNotch target co-incubations, can be evaluated for the ability to recruit primary human T cells across a porous membrane. We have already generated chemokine synNotch response constructs and tested them in anti-CD19 synNotch cells, stably integrated by lentivirus, in which they expressed high levels of chemokine in response to CD19+ target cells, and expect to observe high levels of CD8+ T cell recruitment in response to antigen recognition and TME stimuli matched to the ON-switch adaptor,

### 4. Example 4: Combinatorial Control of Universal Adaptor Cells

200. To create combinatorial adaptors that can activate SNAP receptor signaling in response to combinations of antigens on a cell surface (antigen A AND antigen B) or (antigen A AND (NOT antigen B))(FIG. 20) we can create adaptors with caging groups or linkers that are substrates for bacterial nitro-reductase (NTR) encoded by the nsfB gene or other enzymes, including, but not limited to enzymes adapted from directed enzyme prodrug therapy (DEPT) such as antibody DPET (ADEPT), gene DEPT (GDEPT), Virus DEPT (VDEPT), lectin DEPT (LDEPT), polymer DEPT (PDEPT), and clostridia DEPT (CDEPT) such enzymes including but not limited to carboxypeptidase G2 (CPG2), β-D-glucosidase, carboxylesterase (CE) Horseradish perozidase (HRP), purine nucleoside phosphorylase (PNP), cytochrome P450 (CYP 450)/oxazaphosphorine, cytosine deaminase/5-flurocytosine, and human carboxylestesase (hCE-2). NTR selectively reduces nitro aromatics in an orthogonal manner to the eukaryotic environment and has been extensively used as a tool for activating molecular probes in cells and animals, and clinically in conjunction with pro-drug therapies for cancer. Both logic systems can consist of two adaptor-antibody conjugates, one fused to NTR and one bearing the caged BG 20 that is activated by the reductase or the NTR-cleavable linker 21 that is deactivated (through a self-immol alive 1,6-elimination) in the vicinity of NTR (FIG. 21), For therapeutic applications, antibodies can be administered sequentially, first, the NTR-conjugated enzyme followed by the reactive antibody. The INTR-fused antibody can be at a higher local concentration on antigen A-positive cells triggering cleavage or activation of the antibody-conjugate targeting B on the same cell surface, The SNAP receptor can then react with decaged BG or non-cleaved BG. Overall, this system can dramatically increase ON- vs OFF-target specificity and can allow for enhanced localized targeting of disease-relevant cells based on two antigens and expand the diseases safely treatable by antigen receptor therapy. Finally, we can take advantage of the universal receptor format that allows for rapid screening of new CAR antigens and targeting domains by simply combining the universal CAR with new antibodies, and apply the combinatorial system to validate 20 predicted, clinically-relevant antigen combinations for targeting breast, brain, liver and colorectal cancers by AND and NOT logic.

### a) Design and Validation of AND Gate and NOT Gate Adaptors for Universal SNAP Receptors

201. The nitro-reductase antibody fusions can be created by expressing recombinant NTR protein as a fusion with the SNAPtag enzyme in E. coli, following the design of literature-reported fusion proteins (e.g., with fluorescent proteins) that retained NTR function. In order to maximize the decaging kinetics of the nitroimidazole group, we can use a recently reported, enhanced NTR (eINTR) that shows˜100-fold increase in cellular activity over wild-type NTR. We can use the synthetic adaptor molecules 10, 12, or 14 to conjugate eNTR-SNAPtag to any commercial antibody—generating the antigen A-antibody-NTR system. We do not expect any issues, due to experience in generating antibody-protein (e.g., T cell receptor) conjugates. The adaptors 20 and 21 can be synthesized and used as disclosed herein. For both, we are using a nitroimidazole carbamate as the caging group, as it is a well-established substrate for rapid removal by NTR. We can then conjugate 20 and 21 to anti-HER2 and anti-EGFR antibodies, as we have cell lines in-hand that express none, one, or both antigen combinations to evaluate these.

### b) Analysis of Multi-antigen Boolean Logic Gate SNAP-CAR and SynNotch Signaling and Effector Functions

202. We can first evaluate decaging of the BG and cleavage of the BG-containing linker by pre-treating adaptor conjugates with or without recombinant NTR and assaying for staining of SNAP-CAR T cells. To evaluate the ability of the adaptors to lead to AND and NOT logic gate activation of SNAP CAR and SNAP-synNotch receptors, we can perform co-incubation assays with SNAP cells, ON- and OFF-target cells (with no antigen, EGFR only, HER2-only, or both EGFR and HER2) and antibody combinations and assay for receptor activation after 24 hours. For antibody addition, to mimic the sequential antibody administration to be performed in vivo, which includes diffusion of the first antibody away from antigen negative sites, we can first stain target cells with the NTR-antibody fusion protein, wash cells, and then add the BG-reactive adaptor antibody and SNAP receptor cells. We can also perform the experiment using different doses of adaptors, with the expectation that antigen combination specificity can be observed at low levels of NTR adaptor, but lost at high levels where unbound NTR can decage or cleave BG-adaptor conjugates. After confirming potent activation using model antigens, we can then apply the logic switch adaptor systems to screen novel cancer-targeting antigen combinations. Mackay et al recently applied systematic computational methods to identify new clinically relevant combinations cancer targeting combinations that are currently untested. Screening new antigen combinations is an ideal application of the universal receptors, as we can easily mix and match commercial antibodies with SNAP-CAR T cells or SNAP-synNotch cells, while creating CAR logic receptors using traditional methods would require the laborious process of generating new receptors for each antigen pair. We can evaluate the ten AND and ten (A) AND (NOT B) antigen combinations listed in Table 1 using antigen (+) cell lines identified through the cell line expression database. We can generate combinatorial adaptors and perform co-incubation lysis assays with SNAP-CAR T cells evaluating lysis of targeted cell lines. To mimic single antigen negative conditions, for first pass, we can pre-stain cell lines with unlabeled antibody to block antigen recognition prior to co-incubation assay. We can later create antigen negative cell lines using CRISPR/Cas9 and for antigens controls for NOT combinations, we can generate stable, double(+) antigen cell lines through lentiviral transduction.

TABLE 1
Clinically relevant antigen combinations predicted to confer
cancer specificity vs. normal tissues to be evaluated
combinatorically using adaptors and SNAP receptors

| A AND B | | | A AND (NOT B) | | |
|---|---|---|---|---|---|
| cancer | antigen | antigen | cancer | antigen | antigen |
| type | A | B | type | A | B |
| breast | HTR4 | EDNRB | head and | MUC1 | ABCD3 |
| | | | neck | | |
| colorectal | FAP | ABCC8 | liver | ABCC8 | EXOC1 |
| and prostate | | | | | |
| colorectal | SCN3A | EPCAM | liver | CLCN2 | EXOC1 |
| and prostate | | | | | |
| colorectal | SCN3A | CEA | liver | S1PR5 | ICAM3 |
| glioma | GLRB | CD56 | lung | EPCAM | CMTM6 |
| glioma | GLRB | CD123 | lymphoma | CD19 | ITGAL |
| liver | S1PR5 | ABCC8 | lymphoma | CD19 | CD68 |
| liver | S1PR5 | ABCB1 | stomach | SCTR | CYYR1 |
| ovarian | EDNRB | ABCC8 | stomach | HTR4 | ATP6V1H |
| prostate | ABCC8 | PSMA | thyroid | EPCAM | ABCB9 |

What is claimed is:

1. A conditional universal chimeric antigen receptor (CAR) system, comprising i) a CAR, comprising a receptor that, targets a tag ligand on a conditional adaptor molecule, a hinge domain, and a signaling domain; and ii) a conditional adaptor molecule comprising an antigen recognition element, a stimulus reactive group, and a tag ligand.

2. The conditional universal CAR system of claim 1, wherein the tag ligand on the conditional adaptor molecule comprises a benzylguanine (BG), benzylcytosine (BC), chloroalkane, fluoresceine (FITC), SpyTag, leucine-zipper, La-SS-B, CD19, anti-folate receptor antibody, Fc domain, peptide neoepitope (PNE), or biotin.

3. The conditional universal CAR system of claim 1, wherein the conditional adaptor molecule comprises NHS-ester conjugation, disulfide re-stapling, glycan conjugating chemistry, a recombinant antibody with tag ligand incorporation through one or more short peptide tags, sortase mediated ligation, THIOMABs, chemical ligation, split inteins, and/or unnatural amino acids.

4. The conditional universal CAR system of claim 3, wherein the antigen recognition element comprises an antibody or antigen recognizing fragment thereof.

5. The conditional universal CAR system of claim 4, wherein the antigen recognition element comprises rituximab, FMC63, herceptin, cetuximab, nimotuzumab, panitumumab, omalizumab, tositurnornab, trastuzumab, gemtuzurnab, aletrituzurnab, bevacuzimab or an antigen-binding fragment of any one thereof.

6. The conditional universal CAR system of claim 3, wherein the antigen recognition element comprises a protein binding domain, lectin, DNA aptamer, RNA aptamer, a small molecule ligand for cell surface receptor, or a peptide/protein ligand for natural protein receptor.

7. The conditional universal CAR system of any of claims 1-6, wherein the stimulus to which the stimulus reactive group is reactive comprises a light, enzyme activity, small molecule, pH, H₂O₂, hypoxia, and/or ROS.

8. The conditional universal CAR system of any of claims 1-7, wherein the stimulus reactive group comprises a cleavable linker.

9. The conditional universal CAR system of claim 8, wherein the stimulus reactive group comprises photocleavable or phosphine cleavable linker.

10. The conditional universal CAR system of any of claims 1-9, wherein the stimulus reactive group comprises a stimulus reactive caging group that blocks the CAR from binding to the tag ligand.

11. The conditional universal CAR system of claim 10, wherein the stimulus reactive group comprises a light reactive caging group comprising nitrobenzyl, coumarin, BODIPY, or cyanin.

12. The conditional universal CAR system of any of claims 1-10, wherein the stimulus reactive group comprises a light reactive and wherein wavelength of the light stimulus comprises 365, 405, 544, or 780 nm).

13. The conditional universal CAR system of any of claims 1-7, wherein the stimulus reactive group comprises an enzyme reactive group and the enzyme to which the reactive group is reactive comprises legumain, matrix metalloproteinase, pyridoxal kinase (PDXK,), aldehyde dehydrogenase 7 family, member A1, (ALDH7A1), lipase C, hepatic type (UPC), poly(ADP-ribose) polymerase 1 (PARP1), pyruvate kinase M2 (PKM2), phosphoglycerate kinase 1 (PGK1), ketohexokinase-A (KHK-A), hexokinases (HK), nucleoside diphosphate kinase (NDPK or NDK), and 6-phosphofructo-2-kinase/fructose-2,6-biphosphatase 4 (PFKFB4), mitochondrial α-ketoglutarate dehydrogenase (α-KGDF1), lysine acetyltransferase 2A (KAT2A), acetyl-CoA synthetase short-chain family member 2 (ACSS2), ATP-citrate lyase (ACLY), pyruvate dehydrogenase complex (PDC), α-ketoglutarate dehydrogenase (α-KGDH), CD39, CD73, or fumarase.

14. The conditional universal CAR system of any of claims 1-7, wherein the stimulus reactive group is reactive to a small molecule comprising phosphine or tetrazine.

15. The conditional universal CAR system of any of claims 1-14, further comprising one or more co-stimulation domains.

16. The conditional universal CAR system of claim 15, wherein the one or more co-stimulation domains comprise signaling domains for CD27, CD28, ICOS, 4-1BB, or OX40.

17. The conditional universal CAR system of any of claims 1-15, wherein tag ligand targeting CAR is comprised on a CAR T cell, CAR NK cell, CAR NK T cell, CAR B cell, or CAR macrophage.

18. A conditional universal synthetic Notch (synNotch) receptor, said synNotch receptor comprising a conditional adaptor molecule comprising a stimulus reactive group and a tag, a notch core comprising one or more cleavage sites, and one or more transcription factors.

19. The conditional universal synNotch of claim 18, wherein the conditional adaptor molecule comprises a tag ligand comprising benzylguanine (BG), benzylcytosine (BC), chloroalkane, fluoresceine (FITC), SpyTag, leucine-zipper, La-SS-B, CD19, anti-folate receptor antibody, Fc domain, peptide neoepitope (PNE), or biotin,

20. The conditional universal synNotch of claim 18 or 19, wherein the adaptor molecule comprises NHS-ester conjugation, disulfide re-stapling, glycan conjugating chemistry, a recombinant antibody with tag incorporation through one or more short peptide tags, sortase mediated ligation, chemical ligation, split inteins, THIOMBs, and/or unnatural amino acids.

21. The conditional universal synNotch of any of claims 18-20, wherein the stimulus to which the stimulus reactive group is reactive comprises a light, enzymes, small molecule, pH, hypoxia, H₂O₂, and/or ROS.

22. The conditional universal synNotch of any of claims 18-21, wherein the stimulus reactive group comprises stimulus cleavable linker.

23. The conditional universal synNotch of claim 22, wherein the stimulus reactive group comprises photocleavable or phosphine cleavable linker.

24. The conditional universal synNotch of any of claims 18-21, wherein the stimulus reactive group comprises a stimulus reactive caging group that blocks the receptor from binding to the tag ligand.

25. The conditional universal synNotch of any of claims 18-21, wherein the stimulus reactive group comprises a light reactive caging group consisting of nitrobenzyl, coumarin, BODIPY, or cyanin.

26. The conditional universal synNotch of claim 25, wherein the stimulus reactive group comprises a light reactive and wherein wavelength of the light stimulus comprises 365, 405, 544, or 780 nm.

27. The conditional universal synNotch of any of claims 18-21, wherein the stimulus reactive group comprises an enzyme reactive group and the enzyme to which the reactive group is reactive comprises legumain, matrix metalloproteinase, pyridoxal kinase (PDXK), aldehyde dehydrogenase 7 family, member A1, (ALDH7A1), lipase C, hepatic type (LIPC), poly(ADP-ribose) polymerase 1. (PARP1), pyruvate kinase M2 (PKM2), phosphoglycerate kinase 1. (PGK1), ketohexokinase-A (KHK-A), hexokinases (HK), nucleoside diphosphate kinase (NDPK or NDK), and 6-phosphofructo-2-kinase/fructose-2,6-biphosphatase 4 (PFKFB4), mitochondrial α-ketoglutarate dehydrogenase (α-KGDF1), lysine acetyltransferase 2A (KAT2A), acetyl-CoA synthetase short-chain family member 2 (ACSS2), ATP-citrate lyase (ACLY), pyruvate dehydrogenase complex (PDC), α-ketoglutarate dehydrogenase (α-KGDH), CD39, CD73, or fumarase.

28. The conditional universal synNotch of any of claims 18-21, wherein the stimulus reactive group is reactive to a small molecule comprising phosphine or tetrazine.

29. The conditional universal synNotch of any of claims 18-28, wherein the transcription factor comprises Gal4-VP64, Cal14-VP16, TetR-VP64, or LacI-VP64.

30. The conditional universal synNotch of any of claims 18-29, further comprising an antigen recognition element; wherein the antigen recognition element can become covalently linked to the conditional universal adaptor molecule.

31. The conditional universal synNotch of any of claims 18-30, wherein the antigen recognition element comprises an antibody or antigen recognizing fragment thereof.

32. The conditional universal synNotch of any of claims 18-31, wherein the antigen recognition element comprises rituximab, FMC63, herceptin, cetuximab, nimotuzumab, panitumumab, omalizumab, tositumomab, trastuzumab, gemtuzumab, alemtuzumab, bevacuzimab or an antigen-binding fragment of any one thereof.

33. The conditional universal synNotch of any of claims 18-32, wherein the antigen recognition element comprises a protein binding domain, lectin, DNA aptamer, RNA aptamer, a small molecule ligand for cell surface receptor, or a peptide/protein ligand for natural protein receptor.

34. An engineered cell comprising the conditional universal CAR of any of claims 1-17 and/or the conditional universal synNotch of any of claims 18-33.

35. The engineered cell of claim 34, further comprising a vector comprising a transcriptional response element operatively linked to a promoter driving expression of one or more cell response genes; wherein the one or more of the transcription factors on the synNotch receptor are specific for the transcriptional response element.

36. The engineered cell of claim 34 or 35, wherein the one or more response genes comprise IL-4, IL-10, FASL, IFN-γ, TNF-α, granzyme A. granzyme B, granulysin, and/or perforin.

37. The engineered cell of any of claims 34-36, wherein one or more transcription factors of the conditional universal synNotch receptor activate expression of one or more native cell response genes.

38. The engineered cell of claim 37, wherein the one or more native cell response genes comprise IL-4, IL-10, FASL, IFN-γ, TNF-α, granzyme A. granzyme B, granulysin, and/or perforin.

39. The engineered cell of any of claims 34-36, wherein the cell is an immune cell, a neuron, an epithelial cell, and endothelial cell, or a stem cell.

40. A method of treating disease or disorder in a subject comprising administering to the subject the conditional universal chimeric antigen receptor (CAR) system of any of claims 1-17, conditional SynNotch of any of claims 18-33, and/or the engineered cell of claim 34-39 to the subject; wherein the disease or disorder comprises a cancer, an autoimmune disease, an autoinflammatory disease, a viral infection, a bacterial infection, or a fungal infection.

41. The method of treating a disease or disorder of claim 40, wherein the disease is a cancer selected from the group consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers, testicular cancer, colon cancer, rectal cancer, prostatic cancer, and pancreatic cancer.

42. The method of treating a disease or disorder of claim 40, wherein the disease an automimmune disease selected from the group consisting of Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal emphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MNNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

43. The method of treating a disease or disorder of claim 40, wherein the disease is an autoinflammatory disease selected from the group consisting of asthma, graft versus host disease, allergy, transplant rejection, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)—Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1β (IL-1β) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behcets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID).

44. The method of treating a disease or disorder of claim 40, wherein the disease is a viral infection selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus Rotavirus B, Rotavirus C, Sindbis virus, Simian Immunodeficiency virus, Human T-cell Leukemia virus type-1, Hantavirus, Rubella virus, Simian Immunodeficiency virus, Human immunodeficiency virus type-1, and Human Immunodeficiency virus type-2.

45. The method of treating a disease or disorder of claim 40, wherein the disease is a bacterial infection selected from the group consisting of Mycobaterium tuberculosis, Mycobaterium Bovis, Mycobaterium Bovis strain BCG, BCG substrains, Mycobaterium avium, Mycobaterium intracellular, Mycobaterium africanuni, Mycobaterium kansasii, Mycobaterium marinum, Mycobaterium ulcerans, Mycobaterium avium subspecies paratuberculosis, Nocardia asteroides, Legionella pneumophila, Salmonella typhi, Salmonella enterica, Shigella boydii, Shigella dysenteriae, Shigella sonnei, Shigella flexneri, Yersinia pestis, Pasteurella haemolytic, Pasteurella multocida, Actinobacillus pleuropneumoniae, Listeria monocytogenes, Listeria ivanovii, Brucella abortus, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, Haemophilus influenzae, Haemophilus ducreyi, Clostridium tetani, and Yersinia enterolitica.

46. The method of treating a disease or disorder of claim 40, wherein the disease is a fungal infection selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carnii, Penicillium marneffi, and Alternaria. atternata.

47. The method of treating a disorder or disease of any of claims 40-46 comprising administering to the subject a first conditional universal CAR system of any of claims 1-17 and a second conditional universal CAR system of any of claims 1-17; wherein the first conditional universal CAR system comprises a stimulus reactive group comprising stimulus cleavable linker and the second CAR system comprises a stimulus reactive caging group that blocks the CAR from binding to the tag.

48. The method of treating a disorder or disease of any of claims 40-46, wherein the first and second conditional universal CAR systems are reactive to the same stimulus.

49. The method of treating a disorder or disease of any of claims 40-46, wherein the first and second conditional universal CAR systems are reactive to different stimuli.
